(19) United States
(12) Patent Application Publication
(10) Pub. No.: US 2016/0163993 A1
(43) Pub. Date: Jun. 9, 2016

(54) AMINE DERIVATIVE AND ORGANIC ELECTROLUMINESCENT DEVICE USING THE SAME

(71) Applicant: Samsung Display Co., Ltd., Yongin-si (KR)

(72) Inventor: Hiromi NAKANO, Yokohama (JP)

(21) Appl. No.: 14/954,968

(22) Filed: Nov. 30, 2015

(30) Foreign Application Priority Data
Dec. 9, 2014 (JP) 2014-248609

(51) Int. Cl. H01L 51/00; C07D 307/91; C07D 333/76; C07C 211/56

(52) U.S. Cl. CPC H01L 51/0059; H01L 51/0061; H01L 51/006; C07C 211/56; C07D 307/91; C07D 333/76; H01L 51/5012

(57) ABSTRACT

An organic electroluminescent device may include an anode; an emission layer on the anode; and at least one layer between the anode and the emission layer, wherein the emission layer or the at least one layer includes an amine derivative represented by Formula 1. The organic electroluminescent device using the amine derivative represented by Formula 1 may have improved emission efficiency and emission lifetime.

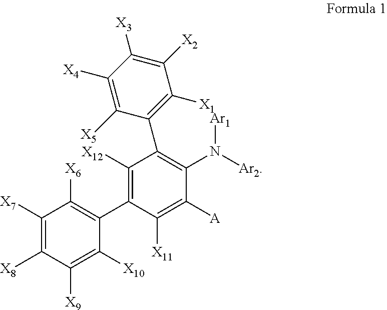

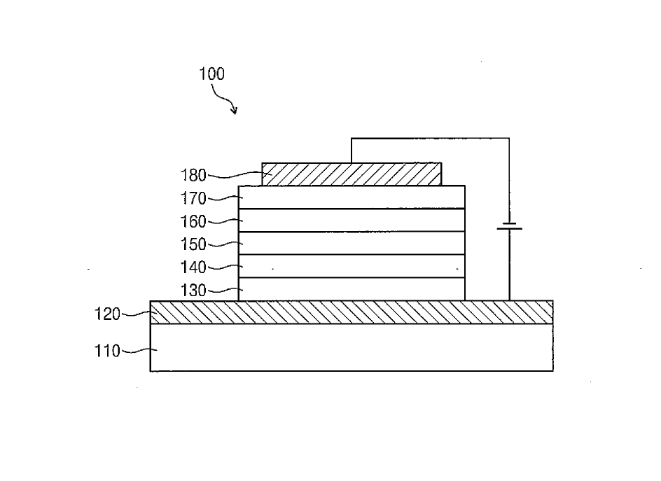

### CROSS-REFERENCE TO RELATED APPLICATION

This patent application claims priority to and the benefit of Japanese Patent Application No. 2014-248609, filed on Dec. 9, 2014, the entire content of which is hereby incorporated by reference.

### BACKGROUND

One or more aspects of embodiments of the present disclosure relate to an amine derivative and an organic electroluminescent device using the same.

Recently, organic electroluminescent (EL) displays using self-emitting organic electroluminescent devices have been actively developed.

A structure of an organic electroluminescent device may, for example, include an anode, a hole injection layer, a hole transport layer, an emission layer, an electron transport layer, an electron injection layer, and a cathode are successively laminated (e.g., in the stated order). Herein, the anode and the cathode may be collectively referred to as “metal layers,” and the hole injection layer, the hole transport layer, the emission layer, the electron transport layer, and the electron injection layer may be collectively referred to as “organic layers.” In such organic electroluminescent device, holes and electrons, respectively injected from the anode and the cathode, are recombined in the emission layer to generate excitons. Light is emitted when the generated excitons transition to the ground state.

To improve the performance of organic electroluminescent devices, various compounds to be used as a material in each of the foregoing layers are being investigated. For example, a compound in which a substituent having a large volume is positioned near an amino group has been disclosed, and a technology by which the lifetime of an organic electroluminescent device may be extended by using the compound has also been disclosed.

However, an organic electroluminescent device using the aforementioned disclosed compound may experience a decrease of emission efficiency. Moreover, in organic electroluminescent devices, compounds that have been disclosed as improving one property, tended to also degrade another property. Therefore, there is a demand for the development of a material compound which may improve the performance of an organic electroluminescent device without degrading other properties.

### SUMMARY

One or more aspects of embodiments of the present disclosure are directed toward a novel amine derivative which may improve the emission efficiency and emission lifetime of the organic electroluminescent device using the amine derivative.

An embodiment of the present disclosure provides an amine derivative represented by the following Formula 1:

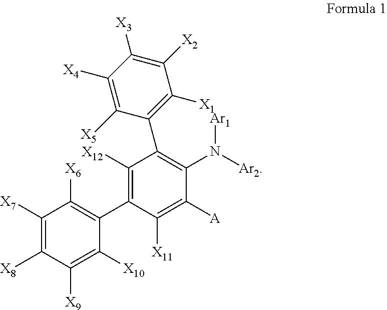

In Formula 1, X₁ to X₁₂ may be each independently selected from hydrogen, deuterium, a halogen atom, a substituted or unsubstituted alkyl group having 1 to 15 carbon atoms, a substituted or unsubstituted aryl group having 6 to 36 carbon atoms for forming a ring, and a substituted or unsubstituted heteroaryl group having 1 to 36 carbon atoms for forming a ring, and a plurality of adjacent groups selected from X₁ to X₁₂ are optionally coupled to each other to form a condensed ring group; Ar₁ and Ar₂ may be each independently selected from a substituted or unsubstituted aryl group having 6 to 36 carbon atoms for forming a ring (the aryl group excluding a pyrenyl group) and a substituted or unsubstituted heteroaryl group having 1 to 36 carbon atoms for forming a ring; and A may be a halogen atom.

The emission efficiency and emission lifetime of the organic electroluminescent device using the amine derivative of the embodiment of the present disclosure may be improved.

In an embodiment, the Ar₁ and Ar₂ may each independently include the aryl group having 6 to 36 carbon atoms for forming a ring, where the aryl group may be selected from a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a benzofluoranthenyl group, a chrysenyl group, a phenylnaphthyl group, a naphthylphenyl group, and a phenylanthracenyl group.

The emission efficiency and emission lifetime of the organic electroluminescent device using the amine derivative of the embodiment of the present disclosure may be further improved.

In an embodiment, the heteroaryl group having 1 to 36 carbon atoms for forming a ring may be selected from a dibenzofuranyl group, a dibenzothienyl group, a carbazolyl group, a dibenzosilolyl group, a phenyldibenzofuranyl group, a phenyldibenzothienyl group, and a phenylcarbazolyl group.

The emission efficiency and emission lifetime of the organic electroluminescent device using the amine derivative of the embodiment of the present disclosure may be improved.

In an embodiment, at least one selected from the Ar₁ and Ar₂ may be selected from a substituted or unsubstituted dibenzofuranyl group, a substituted or unsubstituted dibenzothienyl group, a substituted or unsubstituted carbazolyl group, a substituted or unsubstituted dibenzosilolyl group, and an aryl group substituted with at least one selected from a dibenzofuranyl group, a dibenzothienyl group, a carbazolyl group, and a dibenzosilolyl group.

The emission efficiency and emission lifetime of the organic electroluminescent device using the amine derivative of the embodiment of the present disclosure may be improved.

In an embodiment, the A may be a fluorine atom.

The emission efficiency and emission lifetime of the organic electroluminescent device using the amine derivative of the embodiment of the present disclosure may be further improved.

In an embodiment, the X₁ to X₁₂ may each independently be selected from hydrogen, deuterium, a methyl group, and a phenyl group, and a plurality of adjacent groups selected from X₁ to X₁₂ are optionally coupled to each other to form a condensed ring group.

The emission efficiency and emission lifetime of the organic electroluminescent device using the amine derivative of the embodiment of the present disclosure may be further improved.

An embodiment of the present disclosure provides an organic electroluminescent device, wherein the emission layer includes the amine derivative.

This embodiment may provide an organic electroluminescent device having an improved emission efficiency and emission lifetime.

An embodiment of the present disclosure provides an organic electroluminescent device including at least one layer between the anode and the emission layer, the at least one layer including the amine derivative.

This embodiment may provide an organic electroluminescent device having an improved emission efficiency and emission lifetime.

### BRIEF DESCRIPTION OF THE FIGURES

The accompanying drawing is included to provide a further understanding of the present disclosure, and is incorporated in and constitute a part of this specification. The drawing illustrates example embodiments of the present disclosure and, together with the description, serves to explain principles of the present disclosure. The drawings is a schematic view illustrating an organic electroluminescent device according to an embodiment of the present disclosure.

### DETAILED DESCRIPTION

Hereinafter, example embodiments of the present disclosure will be described in more detail with reference to the accompanying drawing. In the drawing, like reference numerals refer to like elements or elements having like functions throughout the specification, and duplicative explanations thereof will not be provided herein.

### Amine Derivative According to an Embodiment of the Present Disclosure

Hereinafter, description of an amine derivative according to an embodiment of the present disclosure will be provided. An amine derivative according to an embodiment of the present disclosure may be suitable for use as a hole transport material in an organic electroluminescent device, and the amine derivative may be represented by the following Formula 1:

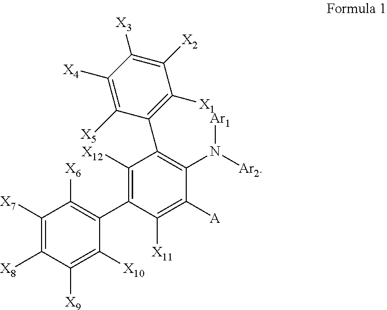

In the above Formula 1, X₁ to X₁₂ may be each independently selected from hydrogen, deuterium, a halogen atom, a substituted or unsubstituted alkyl group having 1 to 15 carbon atoms, a substituted or unsubstituted aryl group having 6 to 36 carbon atoms for forming a ring, and a substituted or unsubstituted heteroaryl group having 1 to 36 carbon atoms for forming a ring, and a plurality of adjacent groups selected from X₁ to X₁₂ may be coupled to each other to form a condensed ring group. As used in the present specification, the statement “atoms for forming a ring” may refer to “ring-forming atoms.”

In Formula 1, Ar₁ and Ar₂ may be each independently selected from a substituted or unsubstituted aryl group having 6 to 36 carbon atoms for forming a ring (the aryl group excluding a pyrenyl group) and a substituted or unsubstituted heteroaryl group having 1 to 36 carbon atoms for forming a ring; and A may be a halogen atom.

Here, in the amine derivative represented by Formula 1, neither of Ar₁, Ar₂, and substituents thereof includes a pyrenyl group. When Ar₁, Ar₂, and/or substituents thereof include a pyrenyl group, the organic electroluminescent device may have a degraded (e.g., decreased) emission efficiency and/or emission lifetime.

Without being bound by any particular theory, it is believed that this degradation of the emission efficiency and emission lifetime of an organic electroluminescent device occurs at least in part because, in an amine derivative including a pyrenyl group, excimers may be formed during the operation of the organic electroluminescent device through interaction between molecules of the pyrenyl group, and thus an energy transfer between molecules may occur. For example, when an amine derivative having a pyrenyl group is used as a hole transport material, the resulting excimer formation may cause an energy transfer between molecules, so that the efficiency of transporting holes into an emission layer may degrade (e.g., decrease), and thus the emission efficiency of the organic electroluminescent device may degrade (e.g., decrease).

In addition, an amine derivative including a pyrenyl group may not be well-suited for use as a lamination material in an organic electroluminescent device, because a large molecular weight of the pyrenyl group may cause degradation (e.g., decrease) of heat resistance, and may hinder film formation through a deposition process. Moreover, since the amine derivative including a pyrenyl group may have a high electron mobility due to the presence of the pyrenyl group, when the amine derivative including a pyrenyl group is formed as a hole transport layer, the function of preventing or reducing electron diffusion from the emission layer may degrade (e.g., may deteriorate). Based on the aforementioned considerations, it is believed that the amine derivative which includes a pyrenyl group is not well-suited for use as a hole transport material, and may cause the emission lifetime and emission efficiency of an organic electroluminescent device to degrade (e.g., to decrease).

In some embodiments, an aryl group having 6 to 36 carbon atoms for forming a ring, used as, for example, Ar₁ and/or Ar₂, may include, for example, a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a benzofluoranthenyl group, a chrysenyl group, a phenylnaphthyl group, a naphthylphenyl group, and a phenylanthracenyl group. In some embodiments, a heteroaryl group having 1 to 36 carbon atoms for forming a ring, used as, for example, Ar₁ and/or Ar₂, may include, for example, a dibenzofuranyl group, a dibenzothienyl group, a carbazolyl group, a dibenzosilolyl group, a phenyldibenzofuranyl group, a phenyldibenzothienyl group, and a phenylcarbazolyl group.

For example, in Formula 1, at least one selected from Ar₁ and Ar₂ may be selected from a substituted or unsubstituted dibenzofuranyl group, a substituted or unsubstituted dibenzothienyl group, a substituted or unsubstituted carbazolyl group, a substituted or unsubstituted dibenzosilolyl group, and an aryl group substituted with at least one selected from a dibenzofuranyl group, a dibenzothienyl group, a carbazolyl group, and a dibenzosilolyl group. An emission lifetime of an organic electroluminescent device using such amine derivative may be improved.

Without being bound by any particular theory, it is believed that the aforementioned effect is obtained at least in part because, in a substituent which includes a dibenzoheterole moiety (e.g., in a dibenzofuranyl group, a dibenzothienyl group, a carbazolyl group, and/or a dibenzosilolyl group), two benzene rings are cross-linked (e.g., fused) via a heteroatom (e.g., a central ring including a heteroatom) so that a π-conjugated system of the dibenzoheterole moiety is expanded, and radical positive ionic species formed during generation of a carrier may be stabilized. According to embodiments of the present invention, in an amine derivative represented by Formula 1, at least one selected from Ar₁ and Ar₂ may be a substituent which includes a dibenzoheterole ring (e.g., a dibenzoheterole moiety).

In some embodiments, in Formula 1, A may be a fluorine atom. Such amine derivative may improve the emission efficiency of an organic electroluminescent device.

Without being bound by any particular theory, it is believed that the improved emission efficiency of an organic electroluminescent device is achieved at least in part because when A is a fluorine atom, the surrounding volume of the nitrogen atom included in an amino group may be expanded, and the energy level of the amine derivative represented by Formula 1 may become higher. Moreover, when A is a fluorine atom, distance between molecules of the amine derivative represented by Formula 1 may become smaller so that carrier mobility becomes even higher, and thus the emission efficiency of an organic electroluminescent device may be further improved.

In some embodiments, in Formula 1, X₁ to X₁₂ may be each independently any substituent selected from the substituents described herein in connection with X₁ to X₁₂. For example, X₁ to X₁₂ may be selected from hydrogen, deuterium, a methyl group, and a phenyl group, and a plurality of adjacent groups selected from X₁ to X₁₂ may be coupled to each other to form a condensed ring group.

For example, an aryl group having 6 to 36 carbon atoms for forming a ring, which may be used as, for example, any of X₁ to X₁₂, may include a phenyl group, a biphenyl group, a terphenyl group, a naphthyl group, an anthryl group, a phenanthrenyl group, a fluorenyl group, an indenyl group, a pyrenyl group, a fluoranthenyl group, a triphenylenyl group, and/or the like.

In some embodiments, a heteroaryl group having 1 to 36 carbon atoms for forming a ring, which may be used as, for example, any of X₁ to X₁₂, may include, for example, a pyrazinyl group, a pyrrolyl group, a pyridyl group, a pyrimidyl group, a pyridazyl group, a furanyl group, a pyranyl group, a thienyl group, a quinolyl group, an isoquinolyl group, a benzofuranyl group, a benzothienyl group, an indolyl group, a benzoxazolyl group, a benzothiazolyl group, a quinoxalyl group, a benzoimidazolyl group, a pyrazolyl group, a tetrazolyl group, an imidazolyl group, an oxazolyl group, an isoxazolyl group, a thiazolyl group, an isothiazolyl group, a carbazolyl group, a dibenzofuranyl group, a dibenzothienyl group, and/or the like.

In some embodiments, an alkyl group having 1 to 15 carbon atoms, which may be used as, for example, any of X₁ to X₁₂, may include, for example, a straight-chain (e.g., linear) alkyl group such as a methyl group, an ethyl group, a propyl group, a butyl group, an octyl group, a decyl group, and/or a pentadecyl group; and a branched chain (e.g., branched) alkyl group such as a t-butyl group.

In some embodiments, in an organic electroluminescent device, an amine derivative represented by Formula 1 may be included in at least one layer disposed between an emission layer and an anode of the organic electroluminescent device. For example, the amine derivative represented by Formula 1 may be included in a hole transport layer and a hole injection layer of an organic electroluminescent device.

However, in an organic electroluminescent device according to embodiments of the present disclosure, a layer which includes the amine derivative represented by Formula 1 is not limited by the above embodiments. For example, the amine derivative represented by Formula 1 may be included in any of the organic layers disposed between an anode and a cathode of an organic electroluminescent device. For example, the amine derivative represented by Formula 1 may be included in an emission layer.

In some embodiments, the amine derivative represented by Formula 1 may be included in a hole transport layer of an organic electroluminescent device having an emission layer which includes a blue emission material or a green emission material. When the amine derivative represented by Formula 1 is included in the hole transport layer of such organic electroluminescent device, the emission efficiency and emission lifetime of the organic electroluminescent device may be improved.

The amine derivative represented by Formula 1 may be selected from compounds 1 to 52. However, the amine derivative according to embodiments of the present disclosure is not limited to the compounds shown below.

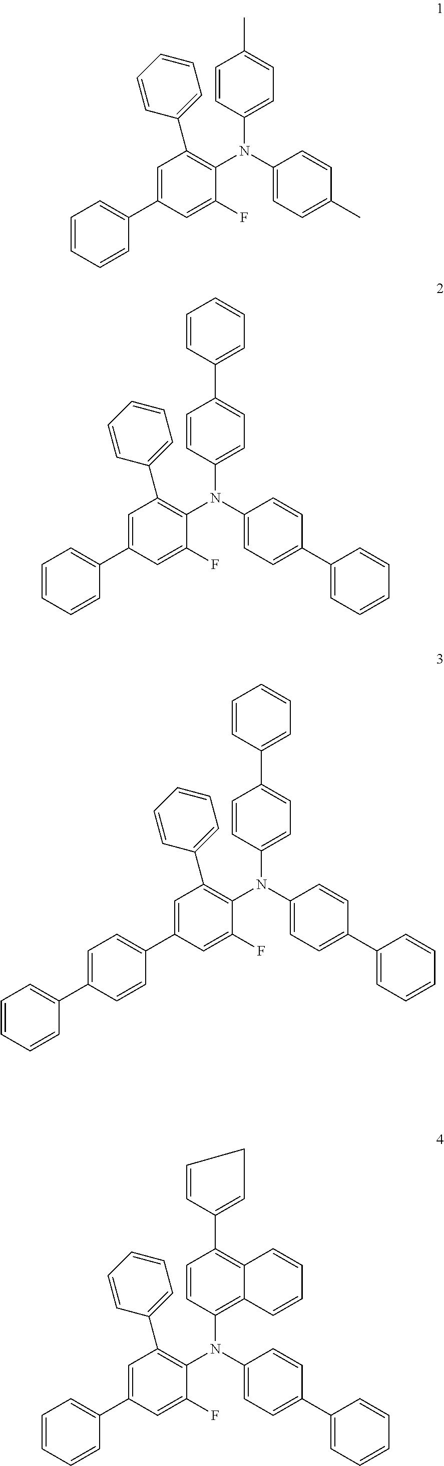 [SMILES: Cc1ccc(N(c2ccc(C)cc2)c2c(F)cc(-c3ccccc3)cc2-c2ccccc2)cc1 ; Fc1cc(-c2ccccc2)cc(-c2ccccc2)c1N(c1ccc(-c2ccccc2)cc1)c1ccc(-c2ccccc2)cc1 ; Fc1cc(-c2ccc(-c3ccccc3)cc2)cc(-c2ccccc2)c1N(c1ccc(-c2ccccc2)cc1)c1ccc(-c2ccccc2)cc1 ; Fc1cc(-c2ccccc2)cc(-c2ccccc2)c1N(c1ccc(-c2ccccc2)cc1)c1ccc(C2=CCC=C2)c2ccccc12]

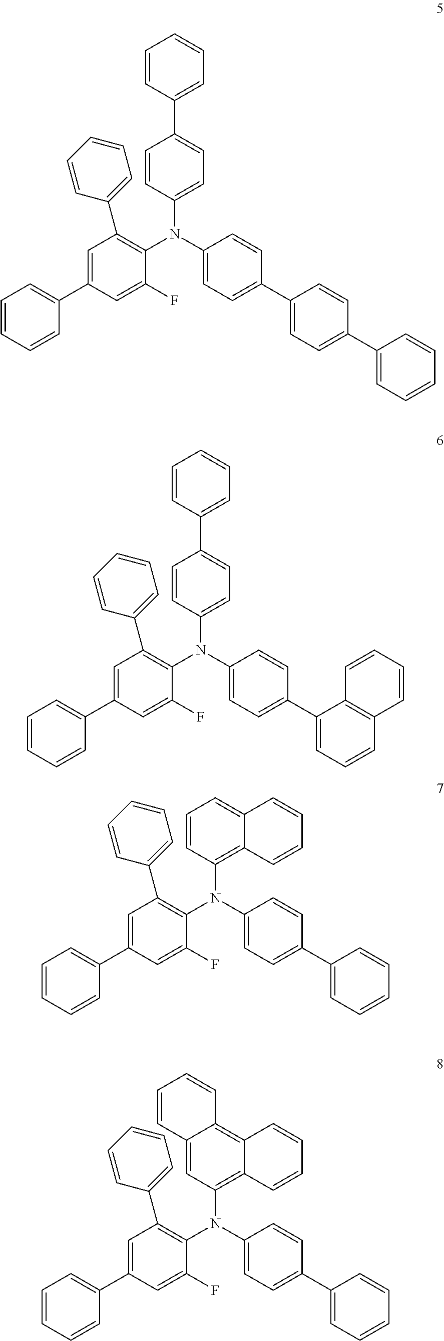 [SMILES: Fc1cc(-c2ccccc2)cc(-c2ccccc2)c1N(c1ccc(-c2ccccc2)cc1)c1ccc(-c2ccc(-c3ccccc3)cc2)cc1 ; Fc1cc(-c2ccccc2)cc(-c2ccccc2)c1N(c1ccc(-c2ccccc2)cc1)c1ccc(-c2cccc3ccccc23)cc1 ; Fc1cc(-c2ccccc2)cc(-c2ccccc2)c1N(c1ccc(-c2ccccc2)cc1)c1cccc2ccccc12 ; Fc1cc(-c2ccccc2)cc(-c2ccccc2)c1N(c1ccc(-c2ccccc2)cc1)c1cc2ccccc2c2ccccc12]

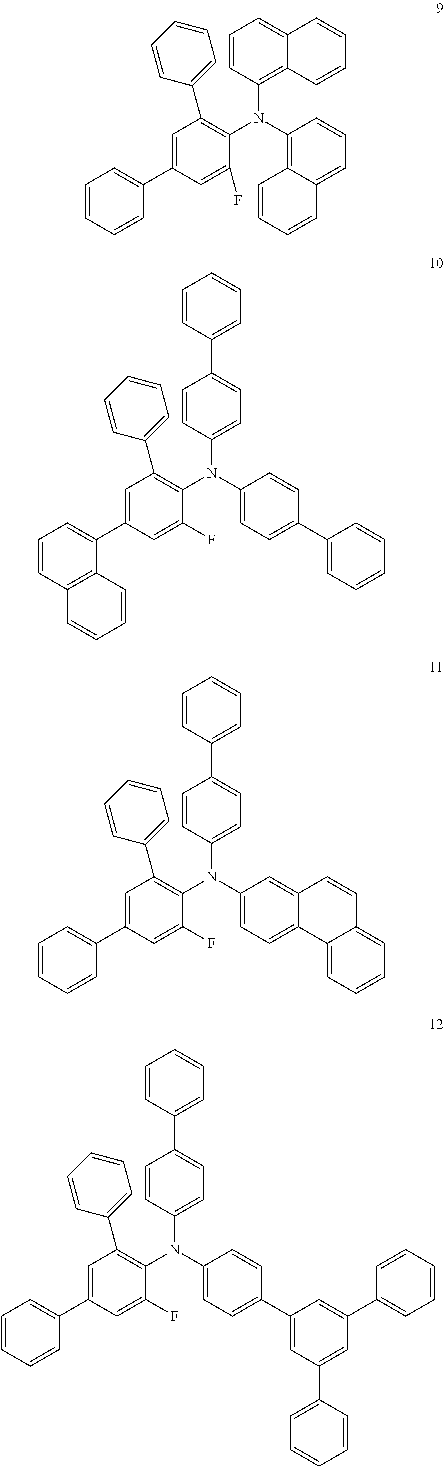 [SMILES: Fc1cc(-c2ccccc2)cc(-c2ccccc2)c1N(c1cccc2ccccc12)c1cccc2ccccc12 ; Fc1cc(-c2cccc3ccccc23)cc(-c2ccccc2)c1N(c1ccc(-c2ccccc2)cc1)c1ccc(-c2ccccc2)cc1 ; Fc1cc(-c2ccccc2)cc(-c2ccccc2)c1N(c1ccc(-c2ccccc2)cc1)c1ccc2c(ccc3ccccc32)c1 ; Fc1cc(-c2ccccc2)cc(-c2ccccc2)c1N(c1ccc(-c2ccccc2)cc1)c1ccc(-c2cc(-c3ccccc3)cc(-c3ccccc3)c2)cc1]

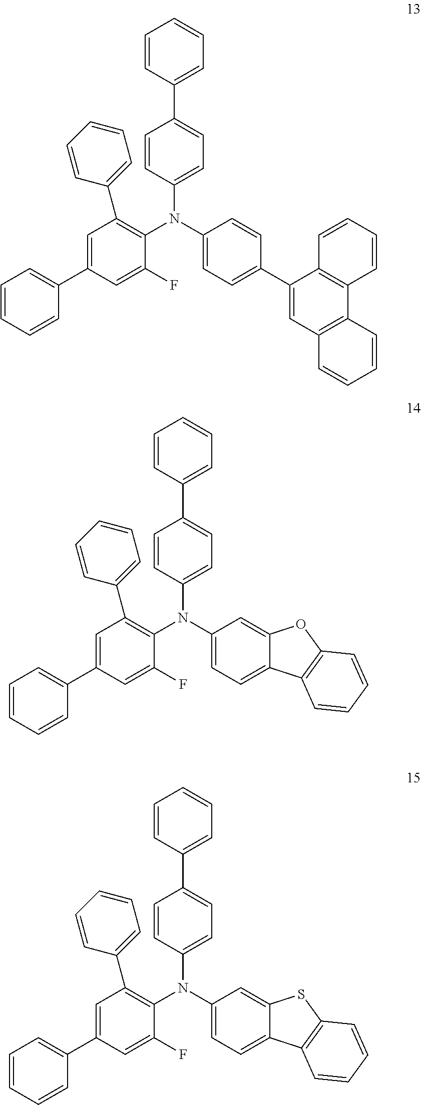 [SMILES: Fc1cc(-c2ccccc2)cc(-c2ccccc2)c1N(c1ccc(-c2ccccc2)cc1)c1ccc(-c2cc3ccccc3c3ccccc23)cc1 ; Fc1cc(-c2ccccc2)cc(-c2ccccc2)c1N(c1ccc(-c2ccccc2)cc1)c1ccc2c(c1)oc1ccccc12 ; Fc1cc(-c2ccccc2)cc(-c2ccccc2)c1N(c1ccc(-c2ccccc2)cc1)c1ccc2c(c1)sc1ccccc12]

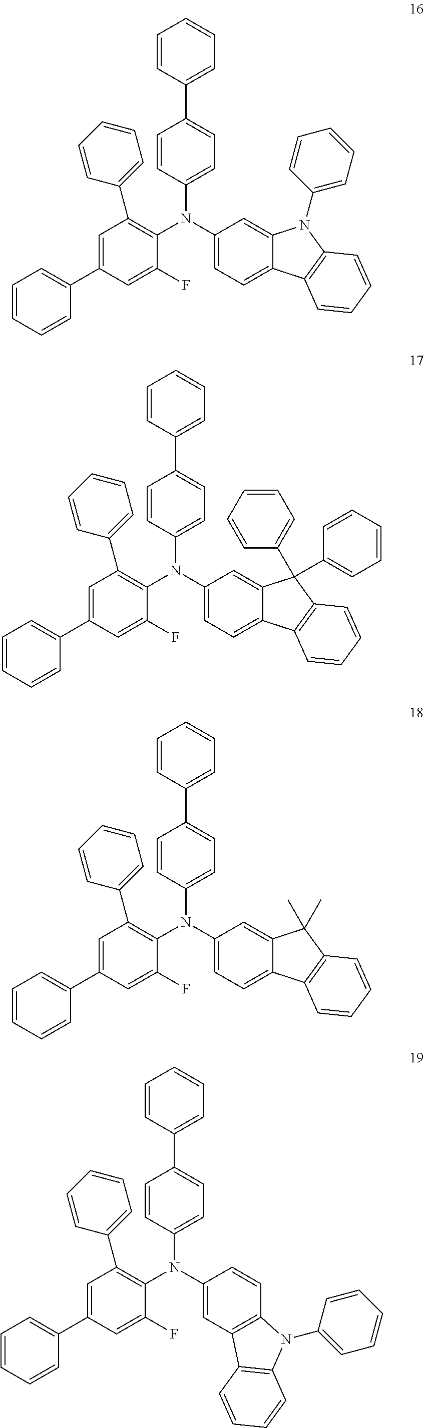 [SMILES: Fc1cc(-c2ccccc2)cc(-c2ccccc2)c1N(c1ccc(-c2ccccc2)cc1)c1ccc2c3ccccc3n(-c3ccccc3)c2c1 ; Fc1cc(-c2ccccc2)cc(-c2ccccc2)c1N(c1ccc(-c2ccccc2)cc1)c1ccc2c(c1)C(c1ccccc1)(c1ccccc1)c1ccccc1-2 ; CC1(C)c2ccccc2-c2ccc(N(c3ccc(-c4ccccc4)cc3)c3c(F)cc(-c4ccccc4)cc3-c3ccccc3)cc21 ; Fc1cc(-c2ccccc2)cc(-c2ccccc2)c1N(c1ccc(-c2ccccc2)cc1)c1ccc2c(c1)c1ccccc1n2-c1ccccc1]

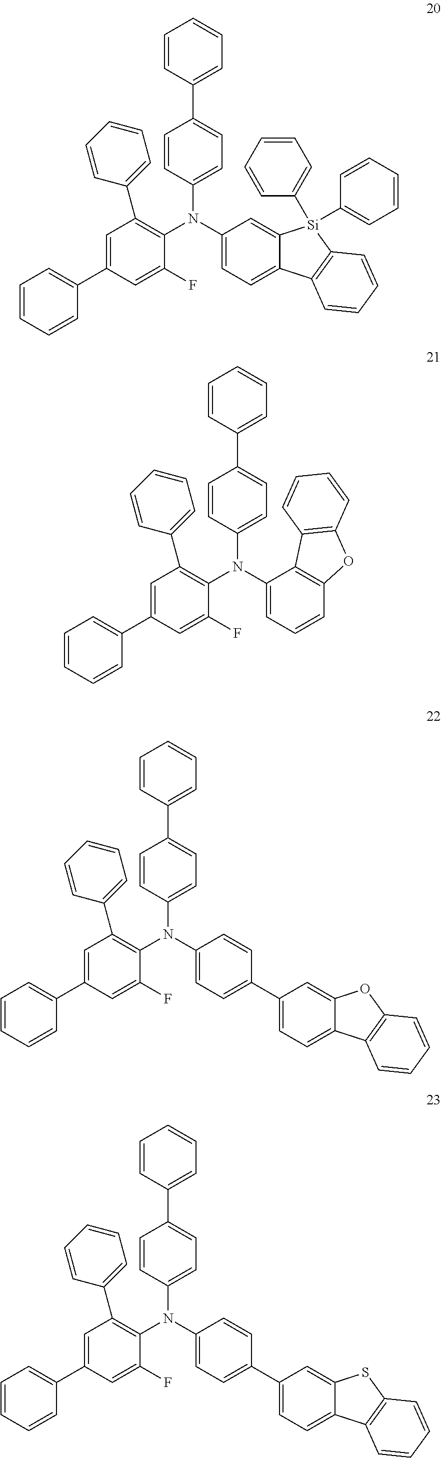 [SMILES: Fc1cc(-c2ccccc2)cc(-c2ccccc2)c1N(c1ccc(-c2ccccc2)cc1)c1ccc2c(c1)[Si](c1ccccc1)(c1ccccc1)c1ccccc1-2 ; Fc1cc(-c2ccccc2)cc(-c2ccccc2)c1N(c1ccc(-c2ccccc2)cc1)c1cccc2oc3ccccc3c12 ; Fc1cc(-c2ccccc2)cc(-c2ccccc2)c1N(c1ccc(-c2ccccc2)cc1)c1ccc(-c2ccc3c(c2)oc2ccccc23)cc1 ; Fc1cc(-c2ccccc2)cc(-c2ccccc2)c1N(c1ccc(-c2ccccc2)cc1)c1ccc(-c2ccc3c(c2)sc2ccccc23)cc1]

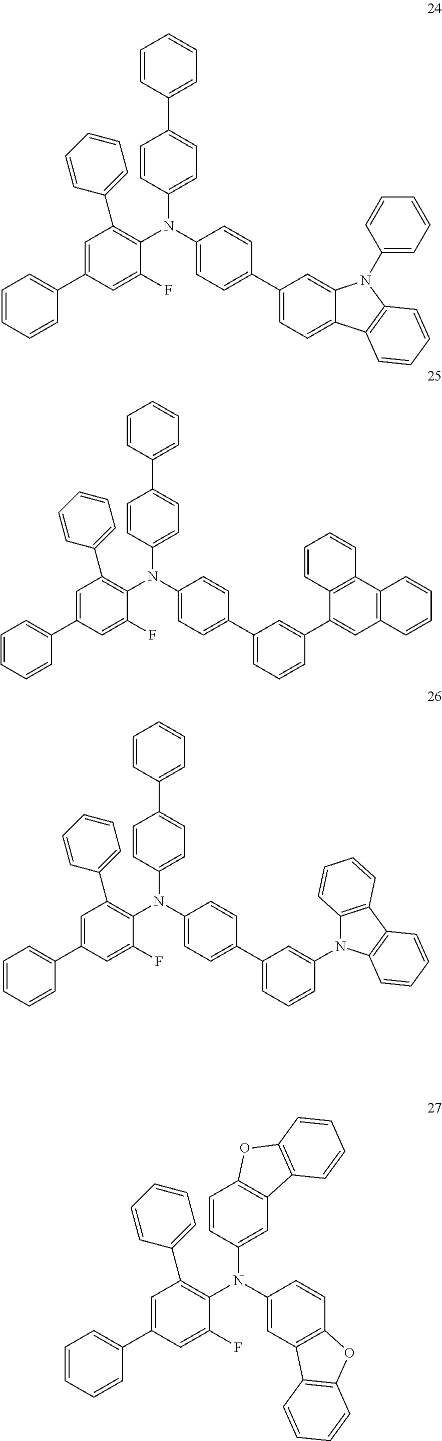 [SMILES: Fc1cc(-c2ccccc2)cc(-c2ccccc2)c1N(c1ccc(-c2ccccc2)cc1)c1ccc(-c2ccc3c4ccccc4n(-c4ccccc4)c3c2)cc1 ; Fc1cc(-c2ccccc2)cc(-c2ccccc2)c1N(c1ccc(-c2ccccc2)cc1)c1ccc(-c2cccc(-c3cc4ccccc4c4ccccc34)c2)cc1 ; Fc1cc(-c2ccccc2)cc(-c2ccccc2)c1N(c1ccc(-c2ccccc2)cc1)c1ccc(-c2cccc(-n3c4ccccc4c4ccccc43)c2)cc1 ; Fc1cc(-c2ccccc2)cc(-c2ccccc2)c1N(c1ccc2oc3ccccc3c2c1)c1ccc2oc3ccccc3c2c1]

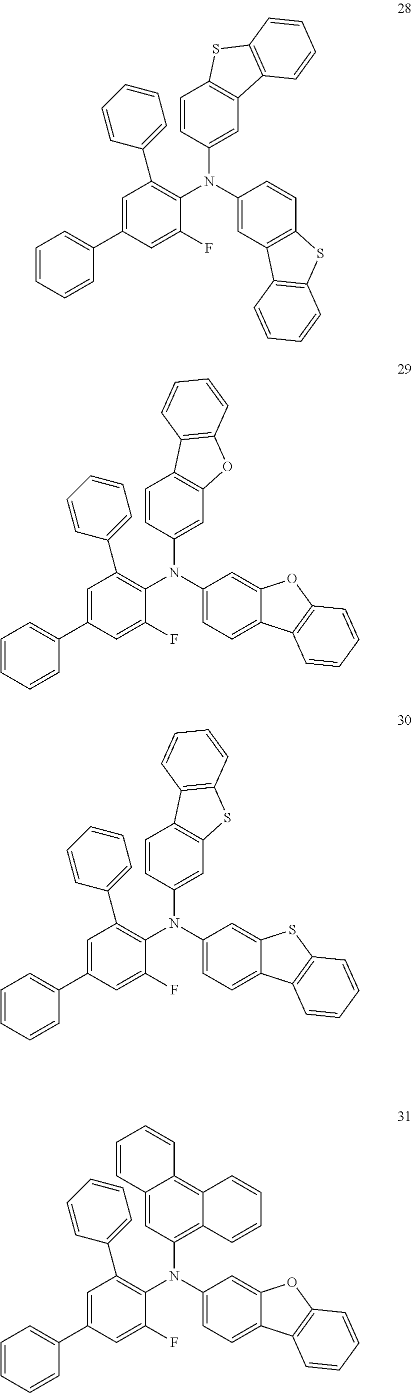 [SMILES: Fc1cc(-c2ccccc2)cc(-c2ccccc2)c1N(c1ccc2sc3ccccc3c2c1)c1ccc2sc3ccccc3c2c1 ; Fc1cc(-c2ccccc2)cc(-c2ccccc2)c1N(c1ccc2c(c1)oc1ccccc12)c1ccc2c(c1)oc1ccccc12 ; Fc1cc(-c2ccccc2)cc(-c2ccccc2)c1N(c1ccc2c(c1)sc1ccccc12)c1ccc2c(c1)sc1ccccc12 ; Fc1cc(-c2ccccc2)cc(-c2ccccc2)c1N(c1ccc2c(c1)oc1ccccc12)c1cc2ccccc2c2ccccc12]

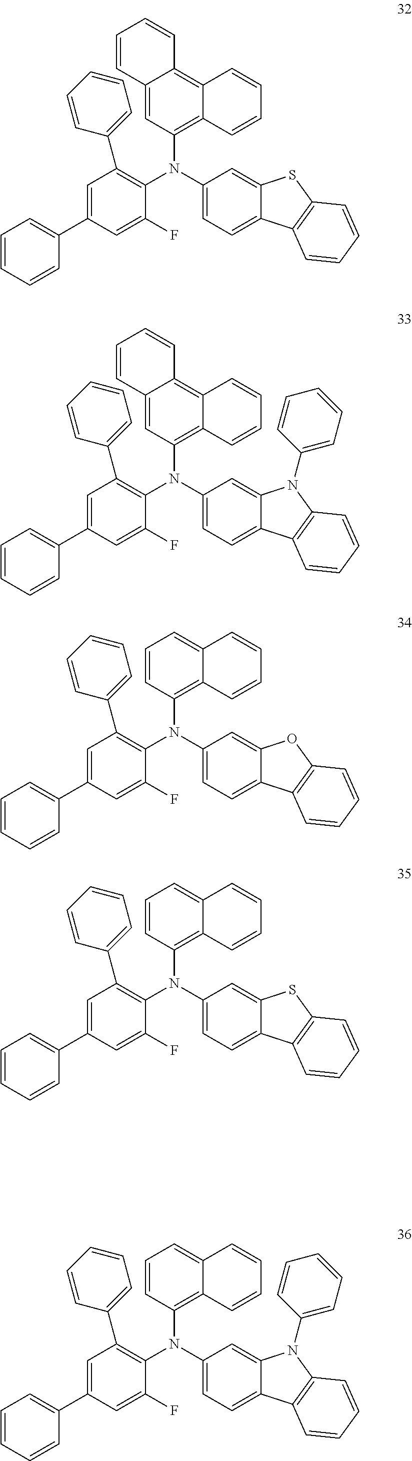 [SMILES: Fc1cc(-c2ccccc2)cc(-c2ccccc2)c1N(c1ccc2c(c1)sc1ccccc12)c1cc2ccccc2c2ccccc12 ; Fc1cc(-c2ccccc2)cc(-c2ccccc2)c1N(c1ccc2c3ccccc3n(-c3ccccc3)c2c1)c1cc2ccccc2c2ccccc12 ; Fc1cc(-c2ccccc2)cc(-c2ccccc2)c1N(c1ccc2c(c1)oc1ccccc12)c1cccc2ccccc12 ; Fc1cc(-c2ccccc2)cc(-c2ccccc2)c1N(c1ccc2c(c1)sc1ccccc12)c1cccc2ccccc12 ; Fc1cc(-c2ccccc2)cc(-c2ccccc2)c1N(c1ccc2c3ccccc3n(-c3ccccc3)c2c1)c1cccc2ccccc12]

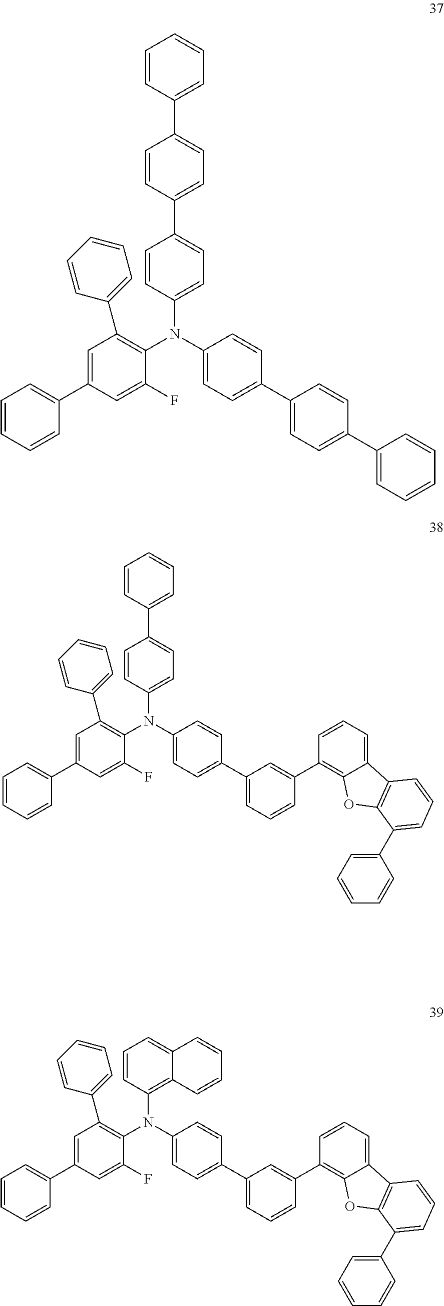 [SMILES: Fc1cc(-c2ccccc2)cc(-c2ccccc2)c1N(c1ccc(-c2ccc(-c3ccccc3)cc2)cc1)c1ccc(-c2ccc(-c3ccccc3)cc2)cc1 ; Fc1cc(-c2ccccc2)cc(-c2ccccc2)c1N(c1ccc(-c2ccccc2)cc1)c1ccc(-c2cccc(-c3cccc4c3oc3c(-c5ccccc5)cccc34)c2)cc1 ; Fc1cc(-c2ccccc2)cc(-c2ccccc2)c1N(c1ccc(-c2cccc(-c3cccc4c3oc3c(-c5ccccc5)cccc34)c2)cc1)c1cccc2ccccc12]

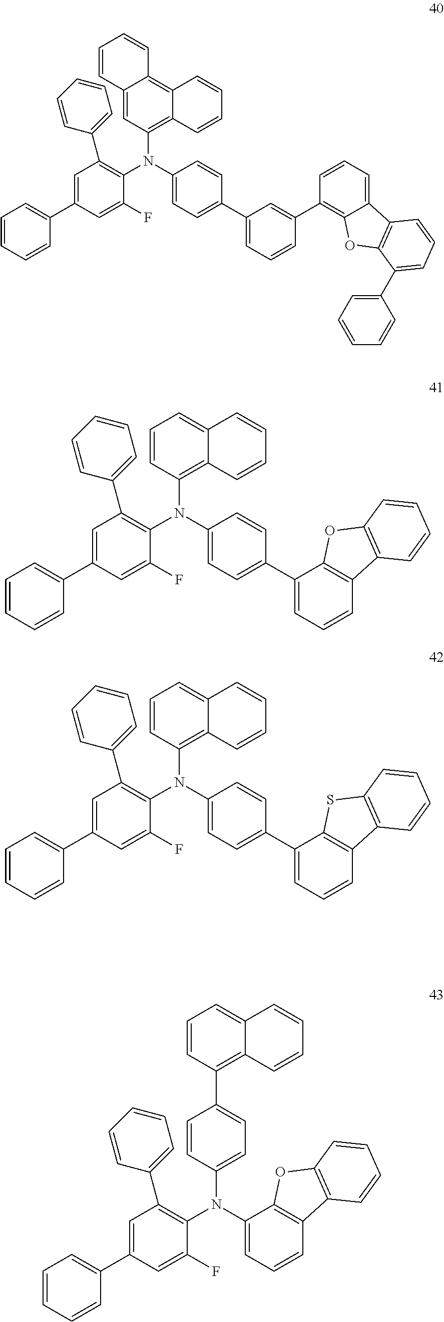 [SMILES: Fc1cc(-c2ccccc2)cc(-c2ccccc2)c1N(c1ccc(-c2cccc(-c3cccc4c3oc3c(-c5ccccc5)cccc34)c2)cc1)c1cc2ccccc2c2ccccc12 ; Fc1cc(-c2ccccc2)cc(-c2ccccc2)c1N(c1ccc(-c2cccc3c2oc2ccccc23)cc1)c1cccc2ccccc12 ; Fc1cc(-c2ccccc2)cc(-c2ccccc2)c1N(c1ccc(-c2cccc3c2sc2ccccc23)cc1)c1cccc2ccccc12 ; Fc1cc(-c2ccccc2)cc(-c2ccccc2)c1N(c1ccc(-c2cccc3ccccc23)cc1)c1cccc2c1oc1ccccc12]

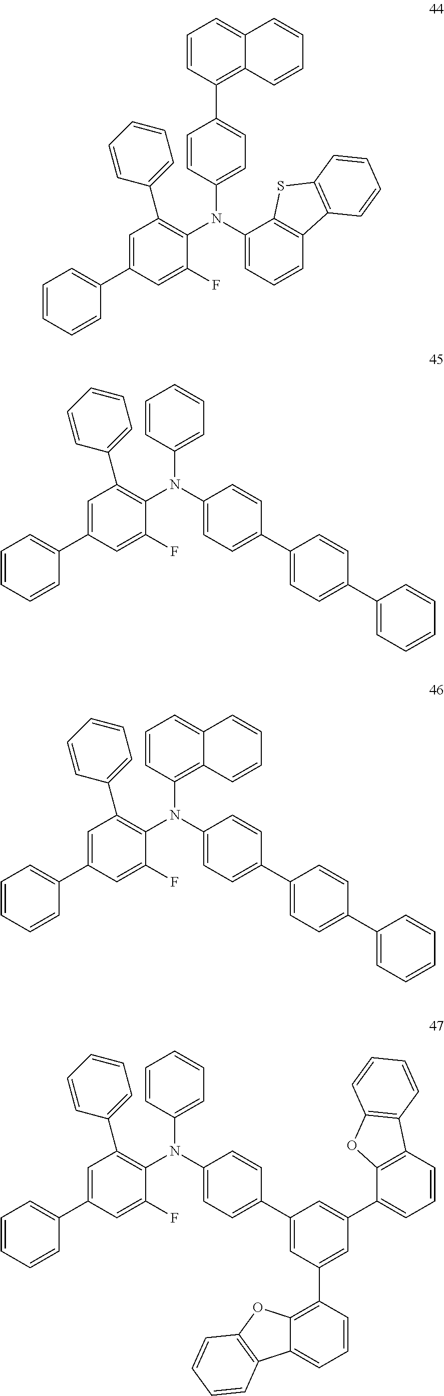 [SMILES: Fc1cc(-c2ccccc2)cc(-c2ccccc2)c1N(c1ccc(-c2cccc3ccccc23)cc1)c1cccc2c1sc1ccccc12 ; Fc1cc(-c2ccccc2)cc(-c2ccccc2)c1N(c1ccccc1)c1ccc(-c2ccc(-c3ccccc3)cc2)cc1 ; Fc1cc(-c2ccccc2)cc(-c2ccccc2)c1N(c1ccc(-c2ccc(-c3ccccc3)cc2)cc1)c1cccc2ccccc12 ; Fc1cc(-c2ccccc2)cc(-c2ccccc2)c1N(c1ccccc1)c1ccc(-c2cc(-c3cccc4c3oc3ccccc34)cc(-c3cccc4c3oc3ccccc34)c2)cc1]

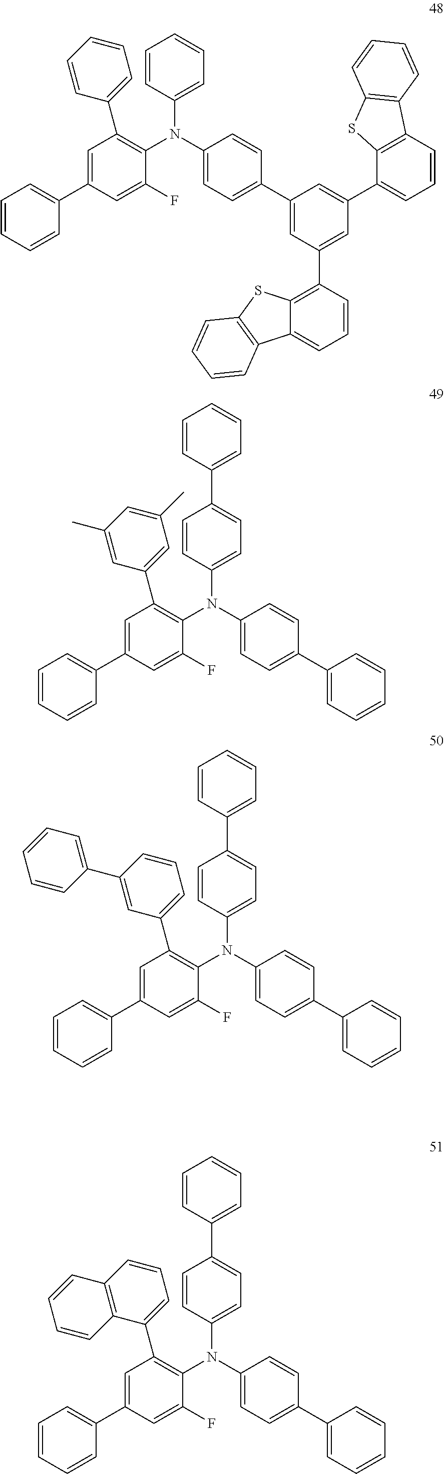

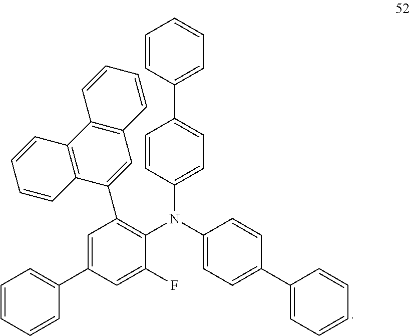 [SMILES: Fc1cc(-c2ccccc2)cc(-c2cc3ccccc3c3ccccc23)c1N(c1ccc(-c2ccccc2)cc1)c1ccc(-c2ccccc2)cc1]

As described above, the amine derivative represented by Formula 1 may be suitable for use as a hole transport material. An organic electroluminescent device that includes the amine derivative represented by Formula 1 as a hole transport material may have an improved emission efficiency and emission lifetime.

Above, an amine derivative according to an embodiment of the present disclosure has been described.

### Organic Electroluminescent Device According to an Embodiment of the Present Disclosure

Hereinafter, an organic electroluminescent device using the amine derivative according to embodiments of the present disclosure will be described in more detail by referring to the drawing. The drawing is a schematic cross-sectional view illustrating an organic electroluminescent device according to an embodiment of the present disclosure.

As illustrated in the drawing, an organic electroluminescent device 100 includes a substrate 110, a first electrode 120 disposed on the substrate 110, a hole injection layer 130 disposed on the first electrode 120, a hole transport layer 140 disposed on the hole injection layer 130, an emission layer 150 disposed on the hole transport layer 140, an electron transport layer 160 disposed on the emission layer 150, an electron injection layer 170 disposed on the electron transport layer 160, and a second electrode 180 disposed on the electron injection layer 170.

For example, the amine derivative of the foregoing embodiment may be included in at least one selected from the hole injection layer 130 and the hole transport layer 140 disposed between the first electrode 120 and the emission layer 150. In some embodiments, the amine derivative according to an embodiment of the present disclosure may also be included in the emission layer 150.

Any suitable substrate capable of being used in an organic electroluminescent device may be used as the substrate 110. For example, the substrate 110 may be a glass substrate, a semiconductor substrate, or a transparent plastic substrate.

The first electrode 120 may be disposed on the substrate 110. For example, the first electrode 120 may be an anode. In some embodiments, the first electrode 120 may be formed of a material having a large work function, such as a metal, an alloy, and/or a conductive compound, and may be formed as a transmissive electrode. For example, the first electrode 120 may be formed using indium tin oxide (In₂O₃—SnO₂:ITO), indium zinc oxide (In₂O₃—ZnO), tin oxide (SnO₂), zinc oxide (ZnO), etc. In some embodiments, the first electrode 120 may also be formed as a reflective electrode in which a material such as magnesium (Mg), aluminum (Al), etc. is laminated on a transmissive electrode formed using the above-described materials.

The hole injection layer 130 may be disposed on the first electrode 120. The hole injection layer 130 may facilitate the injection of holes from the first electrode 120, and may be formed to have a thickness of about 10 nm to about 150 nm.

The hole injection layer 130 may be formed using the amine derivative represented by Formula 1. In some embodiments, the hole injection layer 130 may also be formed using any suitable hole injection material. Non-limiting examples of the material for forming the hole injection layer 130 may include triphenylamine-containing poly ether ketone (TPAPEK), 4-isopropyl-4′-methyldiphenyliodoniumtetrakis(pentafluorophenyl)borate (PPBI), N,N′-diphenyl-N,N′-bis-[4-(phenyl-m-trile-amino)-phenyl]-biphenyl-4,4′-diamine (DNTPD), a phthalocyanine compound such as copper phthalocyanine, 4,4′,4″-tris(3-methylphenylphenylamino)triphenylamine (m-MTDATA), N,N′-di(1-naphthyl)-N,N′-diphenylbenzidine (NPB), 4,4′,4″-tris{N,N-diphenylamino}triphenylamine (TDATA), 4,4′,4″-tris(N,N-2-naphthylphenylamino)triphenylamine (2-TNATA), polyaniline/dodecylbenzenesulfonic acid (PANI/DBSA), poly(3,4-ethylenedioxythiophene)/poly(4-styrenesulfonate (PEDOT/PSS), polyaniline/camphorsulfonic acid (PANI/CSA), polyaniline/poly(4-styrenesulfonate (PANI/PSS), etc.

The hole transport layer 140 may be disposed on the hole injection layer 130. The hole transport layer 140 may facilitate the transporting holes, and may be formed, for example, to have a thickness of about 10 nm to about 150 nm. In some embodiments, the hole transport layer 140 may have a multi-layered structure.

For example, the hole transport layer 140 may be formed using the amine derivative represented by Formula 1 according to embodiments of the present disclosure. In some embodiments, when the amine derivative represented by Formula 1 is included in a layer other than the hole transport layer 140 (e.g., the hole injection layer 130, etc.), the hole transport layer 140 may be formed using any suitable hole transport material. Non-limiting examples of the hole transport material may include 1, 1-bis[(di-4-tolylamino)phenyl]cyclohexane (TAPC); a carbazole derivative such as N-phenyl carbazole and/or polyvinyl carbazole; N,N′-bis(3-methylphenyl)-N,N′-diphenyl-[1,1-biphenyl]-4,4′-diamine (TPD); 4,4′,4″-tris(N-carbazolyl)triphenylamine (TCTA); N,N′-di(1-naphthyl)-N,N′-diphenylbenzidine (NPB), etc.

An emission layer 150 may be disposed on the hole transport layer 140. The emission layer 150 may emit light through fluorescence, phosphorescence, etc., and may be formed, for example, to have a thickness of about 10 nm to about 60 nm. Any suitable light-emitting material may be used as a light-emitting material of the emission layer 150. For example, light-emitting materials such as a fluoranthene derivative, a styryl derivative, a pyrene derivative, an arylacetylene derivative, a fluorene derivative, a perylene derivative, a chrysene derivative, etc. may be used. In some embodiments, the light-emitting material of the emission layer 150 may be selected from a styryl derivative, a pyrene derivative, a perylene derivative, and an anthracene derivative.

For example, an anthracene derivative represented by the following Formula 2 may be used as the light-emitting material of the emission layer 150.

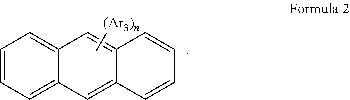

In the above Formula 2,

Ar₃ may be selected from hydrogen, deuterium, a substituted or unsubstituted alkyl group having 1 to 50 carbon atoms for forming a ring, a substituted or unsubstituted cycloalkyl group having 3 to 50 carbon atoms for forming a ring, a substituted or unsubstituted alkoxy group having 1 to 50 carbon atoms for forming a ring, a substituted or unsubstituted aralkyl group having 7 to 50 carbon atoms for forming a ring, a substituted or unsubstituted aryloxy group having 6 to 50 carbon atoms for forming a ring, a substituted or unsubstituted arylthio group having 6 to 50 carbon atoms for forming a ring, a substituted or unsubstituted alkoxycarbonyl group having 2 to 50 carbon atoms for forming a ring, a substituted or unsubstituted aryl group having 6 to 50 carbon atoms for forming a ring, a substituted or unsubstituted silyl group having 5 to 50 carbon atoms for forming a ring, a carboxyl group, a halogen atom, a cyano group, a nitro group, and a hydroxyl group; and n may be an integer from 1 to 10

For example, Ar₃ may be selected from a phenyl group, a biphenyl group, a terphenyl group, a naphthyl group, anthryl group, a phenanthryl group, a fluorenyl group, an indenyl group, a pyrenyl group, an acetonaphthenyl group, a fluoranthenyl group, a triphenylenyl group, a pyridyl group, a furanyl group, a pyranyl group, a thienyl group, a quinolyl group, a isoquinolyl group, a benzofuranyl group, a benzothienyl group, a indolyl group, a carbazolyl group, a benzoxazolyl group, a benzothiazolyl group, a quinoxalyl group, a pyrazolyl group, a dibenzofuranyl group, a dibenzothienyl group, etc. In some embodiments, Ar₃ may be a phenyl group, a biphenyl group, a terphenyl group, a fluorenyl group, a carbazolyl group, a dibenzofuranyl group, a dibenzothienyl group, etc.

The compound represented by the above Formula 2 may be selected from compounds a-1 to a-12. However, the compound represented by Formula 2 is not limited to the following compounds a-1 to a-12.

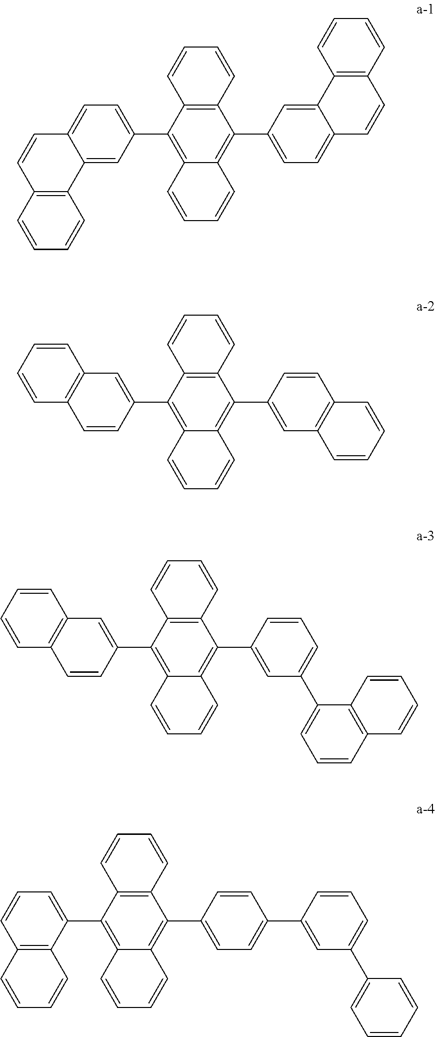

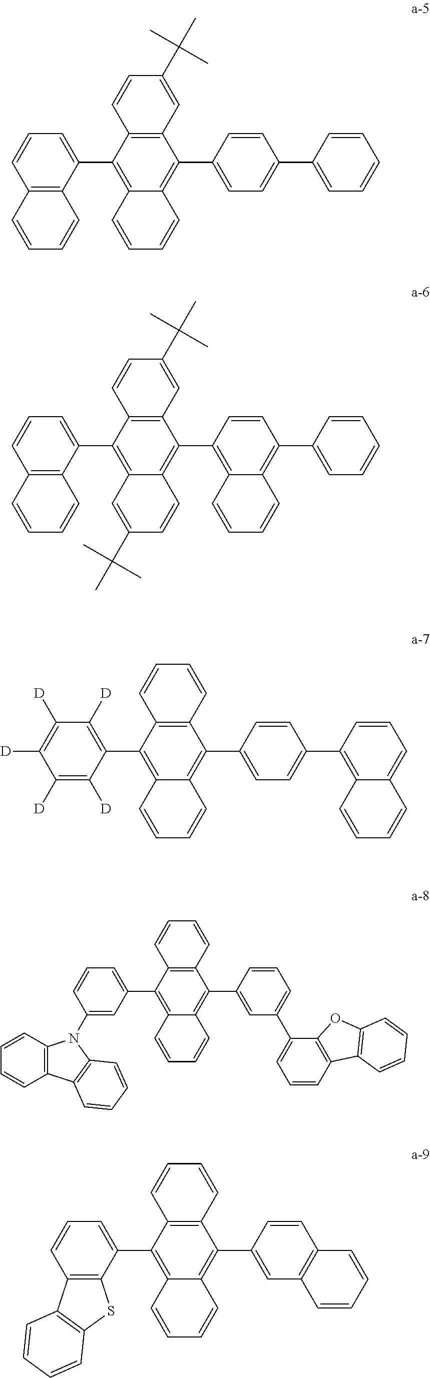

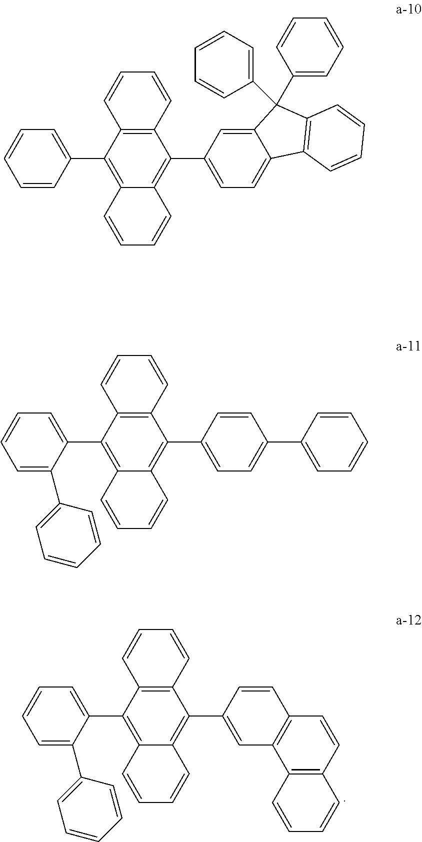

The light-emitting material of the emission layer 150 may include, without limitation, a styryl derivative such as 1,4-bis[2-(3-N-ethylcarbazoryl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi). In some embodiments, the light-emitting material of the emission layer 150 may include a perylene derivative such as 2,5,8,11-tetra-t-butylperylene (TBPe), and/or a pyrene derivative such as 1,1-dipyrene,1,4-dipyrenylbenzene,1,4-bis(N,N-diphenylamino)pyrene. However, embodiments of the present disclosure are not limited to the above example compounds.

The electron transport layer 160 may be disposed on the emission layer 150. The electron transport layer 160 may facilitate the transporting of electrons, and may be formed, for example, to have a thickness of about 15 nm to about 50 nm.

The electron transport layer 160 may be formed using any suitable electron transport material. Non-limiting examples of the electron transport material may include tris(8-hydroxyquinolinato)aluminum (Alq3), a compound having a nitrogen-containing aromatic ring, etc. The compound having a nitrogen-containing aromatic ring may include, for example, a compound having a pyridine ring such as 1,3,5-tri[(3-pyridyl)-phen-3-yl]benzene; a compound having a triazine ring such as 2,4,6-tris(3′-(pyridin-3-yl)biphenyl-3-yl)-1,3,5-triazine; a compound having an imidazole ring such as 2-(4-(N-phenylbenzoimidazolyl-1-ylphenyl)-9,10-dinaphthylanthracene; etc.

The electron injection layer 170 may be disposed on the electron transport layer 160. The electron injection layer 170 may facilitate the injection of electrons from a second electrode 180, and may be formed to have a thickness of about 0.3 nm to about 9 nm. Any material suitable for forming an electron injection layer may be used for the electron injection layer 170. For example, the electron injection layer 170 may be formed using a lithium (Li) complex (e.g., lithium 8-quinolinolato (Liq), lithium fluoride (LiF), etc.), sodium chloride (NaCl), cesium fluoride (CsF), lithium oxide (Li₂O), barium oxide (BaO), etc.

The second electrode 180 may be disposed on the electron injection layer 170. For example, the second electrode 180 may be a cathode. The second electrode 180 may be formed using a material having a small work function, such as a metal, an alloy, and/or a conductive compound, and may be formed as a reflective electrode. The second electrode 180 may also be formed using, for example, a metal such as lithium (Li), magnesium (Mg), aluminum (Al), calcium (Ca), etc., or a mixture of metals such as aluminum-lithium (Al—Li), magnesium-indium (Mg—In), magnesium-silver (Mg—Ag), etc. In some embodiments, the second electrode 180 may be formed to have a thickness of up to about 20 nm, and may be formed as a transmissive electrode using a transparent conductive layer such as indium tin oxide, indium zinc oxide, etc.

According to embodiments of the present disclosure, each of the above-described layers may be formed using one or more suitable film-forming methods, such as vacuum deposition, sputtering, various coating methods, etc., depending on the material used for forming each layer. For example, the organic layers disposed between the first electrode 120 and the second electrode 180 may each independently be formed using one or more suitable deposition methods, coating methods, etc. For example, each of the metal layers (e.g., the first electrode 120 and the second electrode 180) may be formed using, for example, vacuum deposition, sputtering, etc.

Above, the organic electroluminescent device 100 has been described. The organic electroluminescent device 100 according to embodiments of the present disclosure may include the amine derivative represented by Formula 1, thereby realizing an improved emission efficiency and lifetime.

However, a lamination structure of the organic electroluminescent device 100 according to embodiments of the present disclosure is not limited to the above-described embodiments, and the organic electroluminescent device 100 may also be formed to have any suitable lamination structure. For example, the organic electroluminescent device 100 may omit at least one selected from a hole injection layer 130, a hole transport layer 140, an electron transport layer 160, and an electron injection layer 170. In some embodiments, a layer other than the described layers may also be included. Each layer included in the organic electroluminescent device 100 may be formed as a single layer or as multiple layers.

For example, the organic electroluminescent device 100 may further include a hole blocking layer disposed between the hole transport layer 140 and the emission layer 150, in order to prevent or reduce the diffusion of triplet excitons and holes into the electron transport layer 160. The hole blocking layer may be formed using, for example, an oxadiazole derivative, a triazole derivative, a phenanthroline derivative, etc.

### EXAMPLES

Hereinafter, an amine derivative according to an embodiment of the present disclosure and an organic electroluminescent device including the amine derivative will be described with reference to Examples and Comparative Examples. However, the Examples given below are merely example embodiments, and the amine derivative and the organic electroluminescent device including the amine derivative according to embodiments of the present disclosure are not limited to the below-described Examples.

### [Synthesis of Amine Derivative]

A method of synthesizing an amine derivative according to embodiments of the present disclosure will be described in more detail by providing example methods of synthesizing Compound 2, Compounds 14 and 28, and Compound 42. However, the methods described below are merely examples, and methods of synthesizing an organic electroluminescent device according to embodiments of the present disclosure are not limited to the examples given below. The reagents used in connection with Reaction Formulae 1 to 4 were commercially available from Wako Pure Chemical Industries, Ltd., KANTO KAGAKU, and/or TOKYO KASEI KOGYO CO. LTD.

### (Synthesis of Compound 2)

Compound 2 (as an example of the amine derivative according to an embodiment of the present disclosure), was synthesized according to the following Reaction Formula 1.

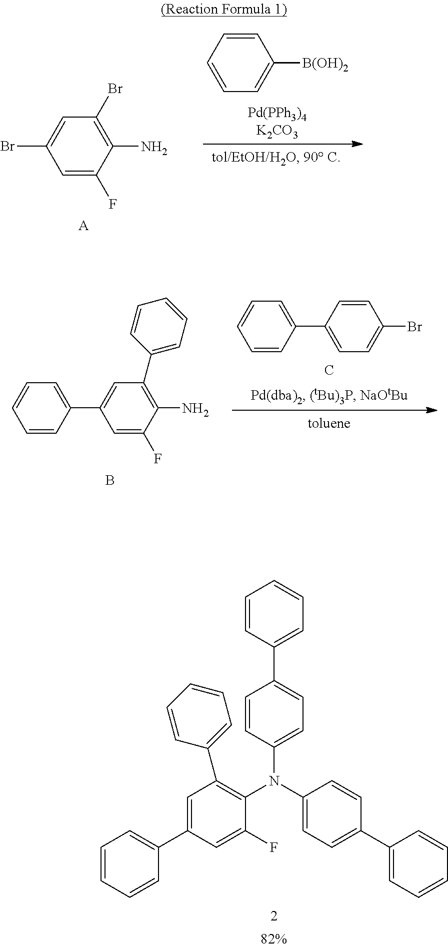

Under an argon (Ar) atmosphere, 40 g of Compound A, 36.2 g of phenylboronic acid, 34 g of tetrakis(triphenylphosphine)palladium(0) (Pd(PPh₃)₄), and 20.5 g of potassium carbonate (K₂CO₃) were mixed in a 1 L, three-necked flask, followed by heating and refluxing the result in a solvent mixture of 740 mL of toluene and 74 mL of a mixture of ethanol and water, for about 8 hours. After air cooling, water was added to the resultant mixture, an organic layer was separated therefrom, and solvent was distilled from the separated organic layer. The crude product thus obtained was purified by silicagel column chromatography (using a solvent mixture of dichloromethane and hexane), and recrystallized using a solvent mixture of toluene and hexane to obtain 37.9 g (Yield 97%) of target Compound B as a white solid.

Under an argon (Ar) atmosphere, 2.0 g of Compound B, 4.0 g of Compound C, 0.49 g of bis(dibenzylideneacetone)palladium(0) (Pd(dba)₂), 0.31 g of tri-tert-butylphosphine ((tBu)₃P), and 2.2 g of sodium tert-butoxide (NaOtBu) were added to a 100 mL, three-necked flask, followed by heating and refluxing the result in 45 mL of toluene for 6 hours. After air cooling, water was added to the resultant mixture, an organic layer was separated therefrom, and solvent was distilled from the separated organic layer. The crude product thus obtained was purified by silicagel column chromatography (using a solvent mixture of dichloromethane and hexane), and recrystallized using a solvent mixture of toluene and hexane to obtain 3.5 g (Yield 82%) of target Compound 2 as a white solid.

The obtained target compound was analyzed using ¹HNMR (1H Nuclear Magnetic Resonance), and the measured chemical shift values were 7.75-7.70 (m, 6H), 7.67 (s,1H), 7.58 (d,1H), 7.55-7.37 (m, 20H), 7.12-7.08 (m, 2H). In addition, the obtained target compound was also analyzed using FAB-MS (Fast Atom Bombardment-Mass Spectrometry), and the measured molecular weight was 567. The results confirmed the obtained compound as being Compound 2.

### (Synthesis of Compound 14)

Compound 14 (as an example of the amine derivative according to an embodiment of the present disclosure) was synthesized according to the following Reaction Formula 2.

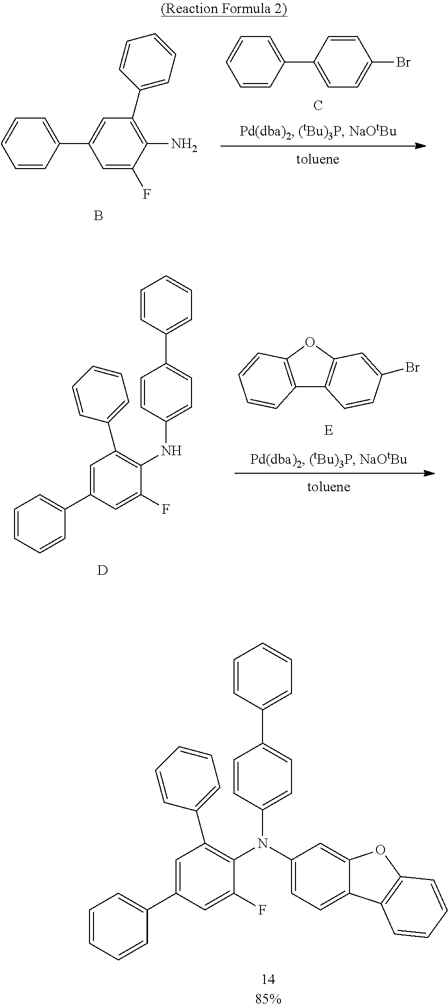

Under an argon (Ar) atmosphere, 2.0 g of Compound B, 4.0 g of Compound C, 0.49 g of bis(dibenzylideneacetone)palladium(0) (Pd(dba)₂), 0.31 g of tri-tert-butylphosphine ((tBu)₃P), and 2.2 g of sodium tert-butoxide (NaOtBu) were added to a 100 mL, three-necked flask, followed by heating and refluxing the result in 45 mL of toluene for 6 hours. After air cooling, water was added to the resultant mixture, an organic layer was separated therefrom, and solvent was distilled from the separated organic layer. The crude product thus obtained was purified by silicagel column chromatography (using a solvent mixture of dichloromethane and hexane), and recrystallized using a solvent mixture of toluene and hexane to obtain 2.8 g (Yield 90%) of target Compound D as a white solid.

Under an argon (Ar) atmosphere, 2.0 g of Compound D, 1.4 g of Compound E, 0.31 g of bis(dibenzylideneacetone)palladium(0) (Pd(dba)₂), 0.19 g of tri-tert-butylphosphine ((tBu)₃P), and 1.4 g of sodium tert-butoxide (NaOtBu) were added to a 100 mL, three-necked flask, followed by heating and refluxing the result in 30 mL of toluene for 6 hours. After air cooling, water was added to the resultant mixture, an organic layer was separated therefrom, and solvent was distilled from the separated organic layer. The crude product thus obtained was purified by silicagel column chromatography (using a solvent mixture of dichloromethane and hexane), and recrystallized using a solvent mixture of toluene and hexane to obtain 2.5 g (Yield 88%) of target Compound 14 as a white solid.

The obtained target compound was analyzed using ¹HNMR (1H Nuclear Magnetic Resonance), and the measured chemical shift values were 8.00 (d, 1H), 7.87 (d, 1H), 7.78 (d, 1H), 7.66 (d, 1H), 7.51-7.47 (m, 2H), 7.45-7.36 (m, 9H), 7.32-7.29 (m, 2H), 7.22-7.19 (m, 2H), 7.15-7.12 (m, 3H), 7.04 (dd, 1H), 7.00 (d, 2H). In addition, the obtained target compound was also analyzed using FAB-MS (Fast Atom Bombardment-Mass Spectrometry), and the measured molecular weight was 582. The results confirmed the obtained compound as being Compound 14.

### (Synthesis of Compound 28)

Compound 28 (as an example of the amine derivative according to an embodiment of the present disclosure) was synthesized according to the following Reaction Formula 3.

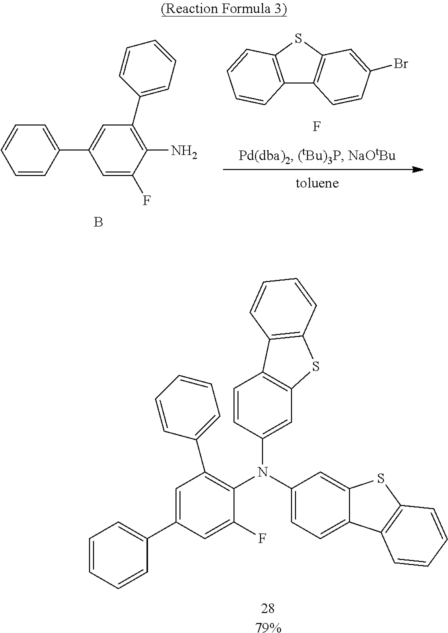

Under an argon (Ar) atmosphere, 2.0 g of Compound B, 4.7 g of Compound F, 0.31 g of bis(dibenzylideneacetone)palladium(0) (Pd(dba)₂), 0.19 g of tri-tert-butylphosphine ((tBu)₃P), and 1.4 g of sodium tert-butoxide (NaOtBu) were added to a 100 mL, three-necked flask, followed by heating and refluxing the result in 45 mL of toluene for 6 hours. After air cooling, water was added to the resultant mixture, an organic layer was separated therefrom, and solvent was distilled from the separated organic layer. The crude product thus obtained was purified by silicagel column chromatography (using a solvent mixture of dichloromethane and hexane), and recrystallized using a solvent mixture of toluene and hexane to obtain 3.7 g (Yield 79%) of target Compound 28 as a white solid.

The obtained target compound was analyzed using ¹HNMR (1H Nuclear Magnetic Resonance), and the measured chemical shift values were 8.01 (d, 1H), 7.80 (d, 1H), 7.78 (d, 1H), 7.75 (d, 1H), 7.51-7.47 (m, 2H), 7.45-7.36 (m, 7H), 7.34-7.30 (m, 2H), 7.26-7.22 (m, 2H), 7.10-7.06 (m, 3H), 7.05 (dd, 1H), 6.99 (d, 2H). In addition, the obtained target compound was also analyzed using FAB-MS (Fast Atom Bombardment-Mass Spectrometry), and the measured molecular weight was 628. The results confirmed the obtained compound as being Compound 28.

### (Synthesis of Compound 42)

Compound 42 (as an example of the amine derivative according to an embodiment of the present disclosure) was synthesized according to the following Reaction Formula 4.

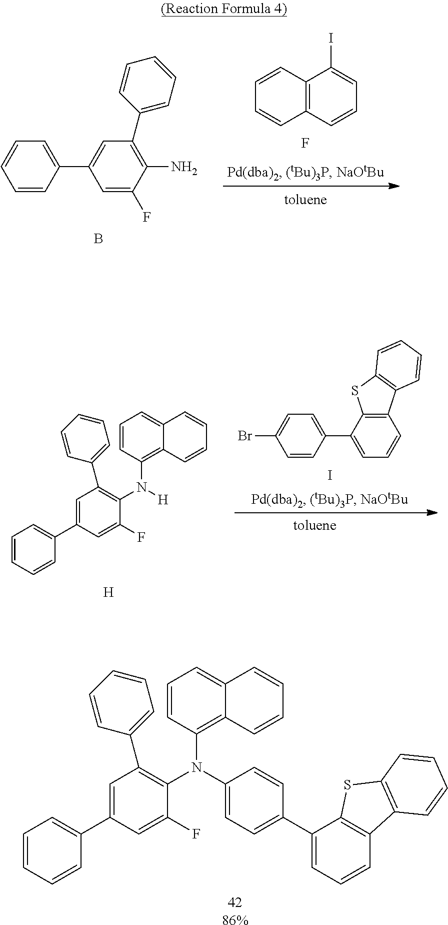

Under an argon (Ar) atmosphere, 2.0 g of Compound B, 1.9 g of Compound G, 0.49 g of bis(dibenzylideneacetone)palladium(0) (Pd(dba)₂), 0.31 g of tri-tert-butylphosphine ((tBu)₃P), and 2.2 g of sodium tert-butoxide (NaOtBu) were added to a 100 mL, three-necked flask, followed by heating and refluxing the result in 45 mL of toluene for 6 hours. After air cooling, water was added to the resultant mixture, an organic layer was separated therefrom, and solvent was distilled from the separated organic layer. The crude product thus obtained was purified by silicagel column chromatography (using a solvent mixture of dichloromethane and hexane), and recrystallized using a solvent mixture of toluene and hexane to obtain 2.8 g (Yield 95%) of target Compound H as a white solid.

Under an argon (Ar) atmosphere, 2.0 g of Compound H, 2.1 g of Compound I, 0.33 g of bis(dibenzylideneacetone)palladium(0) (Pd(dba)₂), 0.21 g of tri-tert-butylphosphine ((tBu)₃P), and 1.5 g of sodium tert-butoxide (NaOtBu) were added to a 100 mL, three-necked flask, followed by heating and refluxing the result in 35 mL of toluene for 6 hours. After air cooling, water was added to the resultant mixture, an organic layer was separated therefrom, and solvent was distilled from the separated organic layer. The crude product thus obtained was purified by silicagel column chromatography (using a solvent mixture of dichloromethane and hexane), and recrystallized using a solvent mixture of toluene and hexane to obtain 2.5 g (Yield 86%) of target Compound 42 as a white solid.

The obtained target compound was analyzed using ¹HNMR (1H Nuclear Magnetic Resonance), and the measured chemical shift values were 8.55 (d, 1H), 8.45 (d, 1H), 8.32 (d, 1H), 8.22 (d, 1H), 8.15 (d, 1H), 7.93 (d, 1H), 7.81-7.37 (m, 22H), 7.08-7.05 (m, 2H). In addition, the obtained target compound was also analyzed using FAB-MS (Fast Atom Bombardment-Mass Spectrometry), and the measured molecular weight was 648. The results confirmed the obtained compound as being Compound 42.

### [Manufacturing Organic Electroluminescent Device]

A blue light-emitting organic electroluminescent device including the amine derivative according to embodiments of the present disclosure was manufactured by vacuum deposition.

### Example 1

After patterning, surface treatment using ultra-violet radiation and ozone (O₃) was performed on an ITO-glass substrate which was subjected to a cleaning treatment. The resulting ITO layer (here, an ITO layer may be a first electrode) on the ITO-glass substrate may have a thickness of about 150 nm. The substrate, on which a surface treatment was completed, was placed inside a deposition apparatus for forming an organic film, and a hole injection layer, a hole transport layer (HTL), an emission layer, and an electron transport layer were successively (e.g., in the stated order) laminated under a vacuum level (e.g., degree of vacuum) of about 10⁻⁴ to about 10⁻⁵ Pa.

The hole injection layer was formed using 4,4′,4″-tris(N,N-2-naphthylphenylamino)triphenylamine (2-TNATA) to have a thickness of about 60 nm. The hole transport layer (HTL) was formed using the above-described Compound 2 to have a layer thickness of about 30 nm. The emission layer was formed using 9,10-di(2-naphthyl)anthracene as a host material and 2,5,8,11-tetra-t-butylperylene (TBP) as a dopant material, to have a layer thickness of about 25 nm. A doping amount of the dopant material was about 3% by mass based on the total mass of the host material. The electron transport material was formed using Alq3 to have a layer thickness of about 25 nm.

Next, the substrate was moved to the deposition apparatus for forming a metal layer, and an electron injection layer and a second electrode were deposited under a vacuum level (e.g., degree of vacuum) of about 10⁻⁴ to 10⁻⁵ Pa, thus manufacturing an organic electroluminescent device. The electron injection layer was formed using lithium fluoride (LiF) to have a layer thickness of about 1 nm, and the second electrode was formed using aluminum (Al) to have a layer thickness of about 100 nm.

### Example 2

An organic electroluminescent device was manufactured through the same (or substantially the same) method as the manufacturing method of Example 1 except that a hole transport layer was formed using Compound 14.

### Example 3

An organic electroluminescent device was manufactured through the same (or substantially the same) method as the manufacturing method of Example 1 except that a hole transport layer was formed using Compound 28.

### Example 4

An organic electroluminescent device was manufactured through the same (or substantially the same) method as the manufacturing method of Example 1 except that a hole transport layer was formed of Compound 42.

Compounds 2, 14, 28, and 42 are illustrated below.

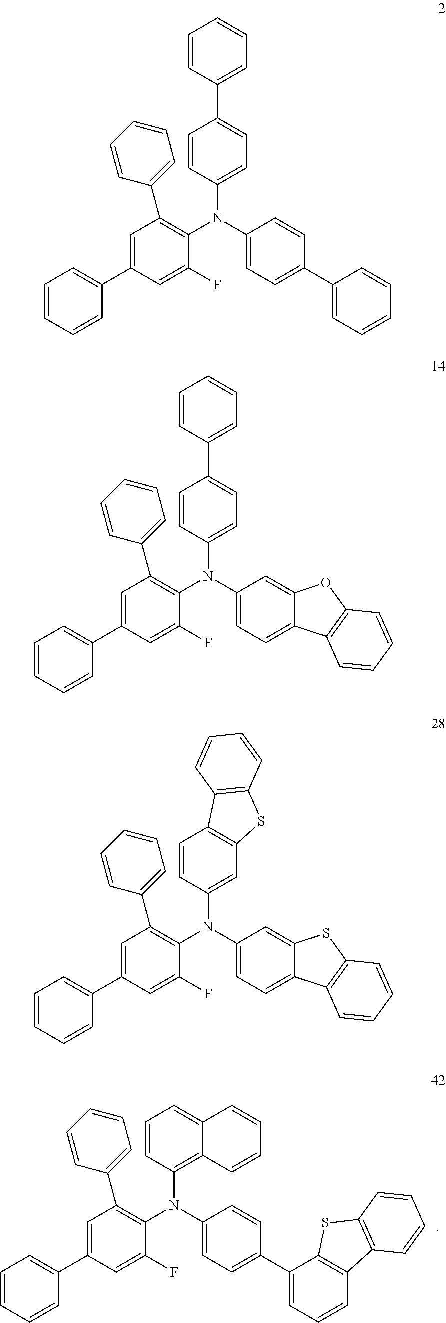 [SMILES: Fc1cc(-c2ccccc2)cc(-c2ccccc2)c1N(c1ccc(-c2ccccc2)cc1)c1ccc(-c2ccccc2)cc1 ; Fc1cc(-c2ccccc2)cc(-c2ccccc2)c1N(c1ccc(-c2ccccc2)cc1)c1ccc2c(c1)oc1ccccc12 ; Fc1cc(-c2ccccc2)cc(-c2ccccc2)c1N(c1ccc2c(c1)sc1ccccc12)c1ccc2c(c1)sc1ccccc12 ; Fc1cc(-c2ccccc2)cc(-c2ccccc2)c1N(c1ccc(-c2cccc3c2sc2ccccc23)cc1)c1cccc2ccccc12]

### Comparative Example 1

An organic electroluminescent device was manufactured through the same (or substantially the same) method as the manufacturing method of Example 1 except that a hole transport layer was formed using Compound 53. Compound 53 is an arylamine derivative generally used as a hole transport material.

### Comparative Example 2

An organic electroluminescent device was manufactured through the same (or substantially the same) method as the manufacturing method of Example 1 except that a hole transport layer was formed using Compound 54. Compound 54 differs from the amine derivative represented by Formula 1 in that A is a phenyl group, instead of a halogen atom.

### Comparative Example 3

An organic electroluminescent device was manufactured through the same (or substantially the same) method as the manufacturing method of Example 1 except that a hole transport layer was formed using Compound 55. Compound 55 is an amine derivative which includes a pyrenyl group as one of the substituents.

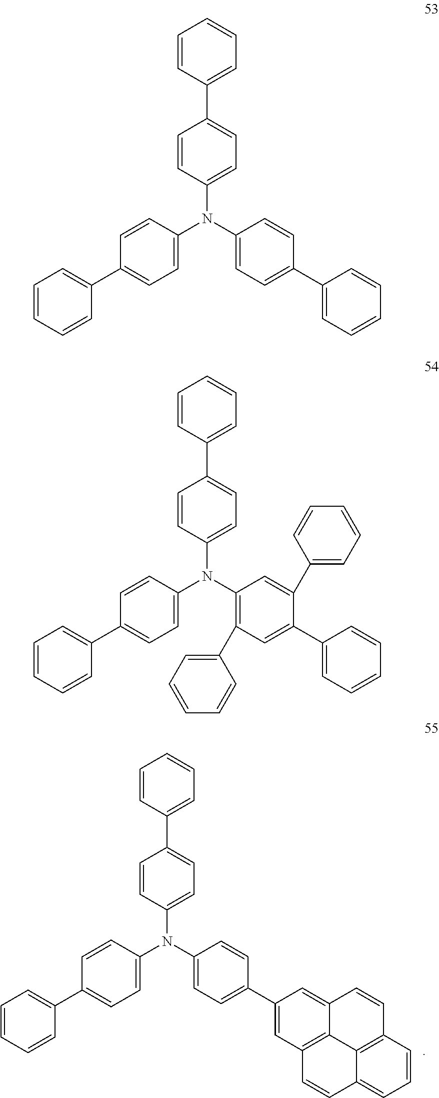

### [Evaluation Results]

Evaluation results of the organic electroluminescent devices manufactured according to Examples 1 to 4 and Comparative Examples 1 to 3 are shown in Table 1. A C9920-11 luminance distribution characteristic measuring device (produced by HAMAMATSU Photonics) was used in the evaluation of the electroluminescent properties of the manufactured organic electroluminescent devices. The results shown in Table 1 were obtained at a current density of about 10 mA/cm², and the emission lifetime was represented by the time (LT50) that it took for the initial luminance of about 1,000 cd/m² to decrease to half of the initial amount.

TABLE 1

| | | Emission | Emission |
|---|---|---|---|
| | | Efficiency | Lifetime |
| | HTL | (cd/A) | (LT50) [hrs] |
| Example 1 | Compound 2 | 8.0 | 1,500 |
| Example 2 | Compound 14 | 7.8 | 2,300 |
| Example 3 | Compound 28 | 8.1 | 2,500 |
| Example 4 | Compound 42 | 7.9 | 2,000 |
| Comparative Example 1 | Compound 53 | 5.0 | 1,200 |
| Comparative Example 2 | Compound 54 | 5.8 | 1,400 |
| Comparative Example 3 | Compound 55 | 4.0 | 1,000 |

Referring to the results in Table 1, it can be seen that the organic electroluminescent devices of Examples 1 to 4 had improved emission efficiency and emission lifetime, when compared to the organic electroluminescent devices of Comparative Examples 1 to 3.

For example, when compared with the organic electroluminescent device of Comparative Example 1 using Compound 53 (a common hole transport material) in the hole transport layer, the organic electroluminescent devices of Examples 1 to 4 had an improved emission efficiency and emission lifetime. In addition, when compared to the organic electroluminescent device of Comparative Example 2 using Compound 54 (in which A in Formula 1 is a phenyl group and not a halogen atom) in the hole transport layer, the organic electroluminescent devices of Examples 1 to 4 had an improved emission efficiency. This effect may be at least in part due to the fact that in Examples 1 to 4, A in Formula 1 included a halogen atom that facilitated the decrease in the distance between molecules of the amine derivative of Formula 1, and improved hole mobility. Moreover, when compared with the organic electroluminescent device of Comparative Example 3 using Compound 55 (which has a pyrenyl group as a substituent) in the hole transport layer, the organic electroluminescent devices of Examples 1 to 4 had an improved emission efficiency and emission lifetime. This effect may be at least in part due to the fact that in Examples 1 to 4 using the amine derivative of Formula 1, a formation of excimers through an interaction between molecules did not occur (or occurred to a significantly lesser extent than in Comparative Example 3).

Furthermore, in the organic electroluminescent devices of Examples 2 to 4 respectively using Compounds 14, 28, and 42 (having a dibenzoheterole moiety in which two benzene rings are cross-linked (e.g., fused) via a heteroatom (e.g., a central ring including a heteroatom)) in the hole transport layer, an expanded 7-conjugated system capable of stabilizing radical positive ionic species was realized, and an emission lifetime was improved when compared with Example 1. Therefore, the amine derivative according to embodiments of the present disclosure may preferably include a dibenzoheterole moiety.

As described above, the amine derivative represented by Formula 1 according to embodiments of the present disclosure may have improved emission efficiency and lifetime. Therefore, the amine derivative according to embodiments of the present disclosure may be suitable for use as a material in an organic electroluminescent device, for example, as a hole transport material.

In an embodiment of the present disclosure, an organic electroluminescent device having improved emission efficiency and emission lifetime may be realized.

As used herein, expressions such as “at least one of,” “one of,” “at least one selected from,” and “one selected from,” when preceding a list of elements, modify the entire list of elements and do not modify the individual elements of the list. Further, the use of “may” when describing embodiments of the present invention refers to “one or more embodiments of the present invention.”

In addition, as used herein, the terms “use,” “using,” and “used” may be considered synonymous with the terms “utilize,” “utilizing,” and “utilized,” respectively.

As used herein, the terms “substantially,” “about,” and similar terms are used as terms of approximation and not as terms of degree, and are intended to account for the inherent deviations in measured or calculated values that would be recognized by those of ordinary skill in the art.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The present disclosure is to be considered illustrative and not restrictive, and the appended claims and equivalents thereof are intended to cover all such modifications, enhancements, and other embodiments, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An amine derivative represented by Formula 1: 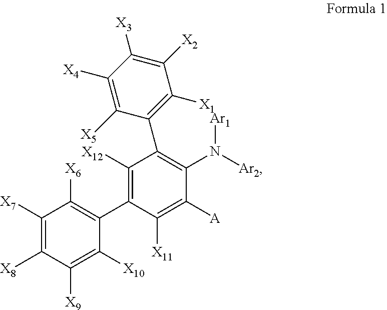
wherein:
X₁ to X₁₂ are each independently selected from hydrogen, deuterium, a halogen atom, a substituted or unsubstituted alkyl group having 1 to 15 carbon atoms, a substituted or unsubstituted aryl group having 6 to 36 carbon atoms for forming a ring, and a substituted or unsubstituted heteroaryl group having 1 to 36 carbon atoms for forming a ring, wherein a plurality of adjacent groups selected from X₁ to X₁₂ are optionally coupled to each other to form a condensed ring group;
Ar₁ and Ar₂ are each independently selected from a substituted or unsubstituted aryl group having 6 to 36 carbon atoms for forming a ring, and a substituted or unsubstituted heteroaryl group having 1 to 36 carbon atoms for forming a ring, the aryl group excluding a pyrenyl group; and
A is a halogen atom.

2. The amine derivative of claim 1, wherein the Ar₁ and Ar₂ each independently comprise the aryl group having 6 to 36 carbon atoms for forming a ring,
wherein the aryl group is selected from the group consisting of a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a benzofluoranthenyl group, a chrysenyl group, a phenylnaphthyl group, a naphthylphenyl group, a phenylanthracenyl group, and combinations thereof.

3. The amine derivative of claim 1, wherein the Ar₁ and Ar₂ each independently comprise the heteroaryl group having 1 to 36 carbon atoms for forming a ring,
wherein the heteroaryl group is selected from the group consisting of a dibenzofuranyl group, a dibenzothienyl group, a carbazolyl group, a dibenzosilolyl group, a phenyldibenzofuranyl group, a phenyldibenzothienyl group, a phenylcarbazolyl group, and combinations thereof.

4. The amine derivative of claim 1, wherein at least one selected from the Ar₁ and Ar₂ is selected from a substituted or unsubstituted dibenzofuranyl group, a substituted or unsubstituted dibenzothienyl group, a substituted or unsubstituted carbazolyl group, a substituted or unsubstituted dibenzosilolyl group, and an aryl group substituted with at least one selected from a dibenzofuranyl group, a dibenzothienyl group, a carbazolyl group, and a dibenzosilolyl group.

5. The amine derivative of claim 1, wherein the A is a fluorine atom.

6. The amine derivative of claim 1, wherein the X₁ to X₁₂ are each independently selected from hydrogen, deuterium, a methyl group, and a phenyl group, wherein a plurality of adjacent groups selected from X₁ to X₁₂ are optionally coupled to each other to form a condensed ring group.

7. The amine derivative of claim 1, wherein the amine derivative represented by Formula 1 is selected from Compounds 1 to 12:

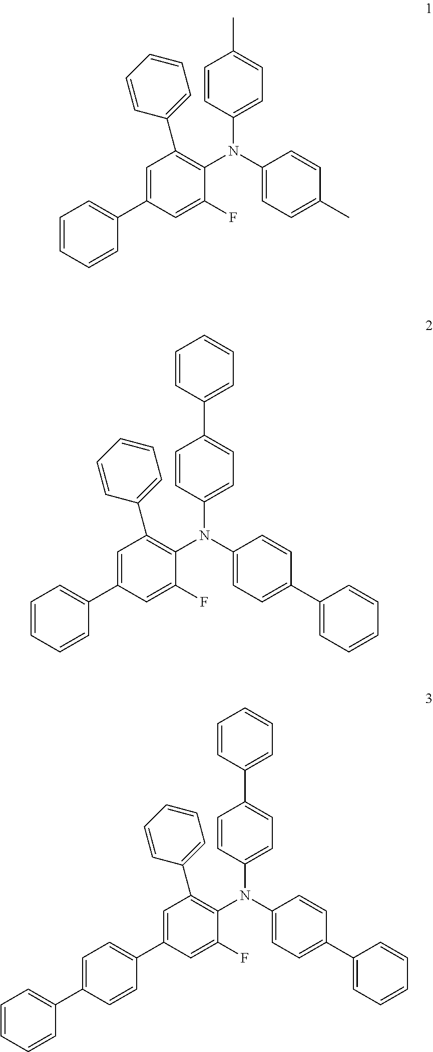 [SMILES: Cc1ccc(N(c2ccc(C)cc2)c2c(F)cc(-c3ccccc3)cc2-c2ccccc2)cc1 ; Fc1cc(-c2ccccc2)cc(-c2ccccc2)c1N(c1ccc(-c2ccccc2)cc1)c1ccc(-c2ccccc2)cc1 ; Fc1cc(-c2ccc(-c3ccccc3)cc2)cc(-c2ccccc2)c1N(c1ccc(-c2ccccc2)cc1)c1ccc(-c2ccccc2)cc1]

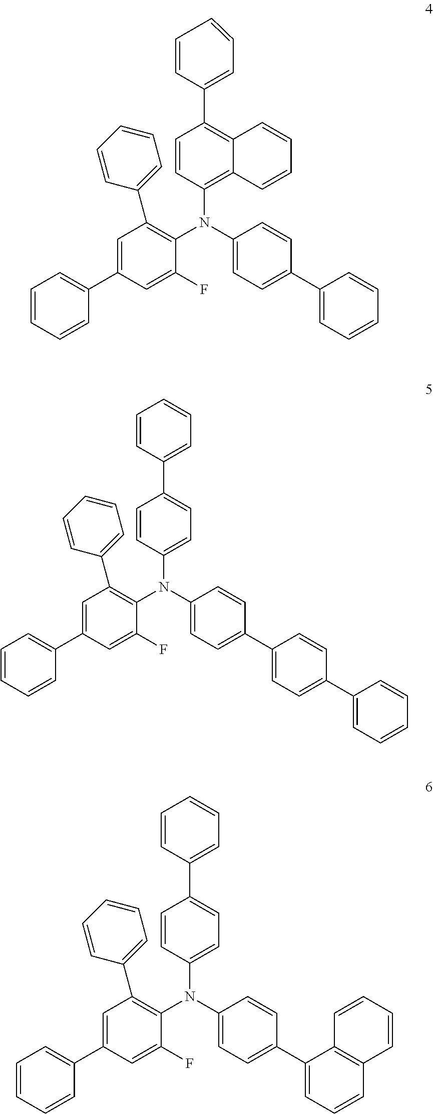 [SMILES: Fc1cc(-c2ccccc2)cc(-c2ccccc2)c1N(c1ccc(-c2ccccc2)cc1)c1ccc(-c2ccccc2)c2ccccc12 ; Fc1cc(-c2ccccc2)cc(-c2ccccc2)c1N(c1ccc(-c2ccccc2)cc1)c1ccc(-c2ccc(-c3ccccc3)cc2)cc1 ; Fc1cc(-c2ccccc2)cc(-c2ccccc2)c1N(c1ccc(-c2ccccc2)cc1)c1ccc(-c2cccc3ccccc23)cc1]

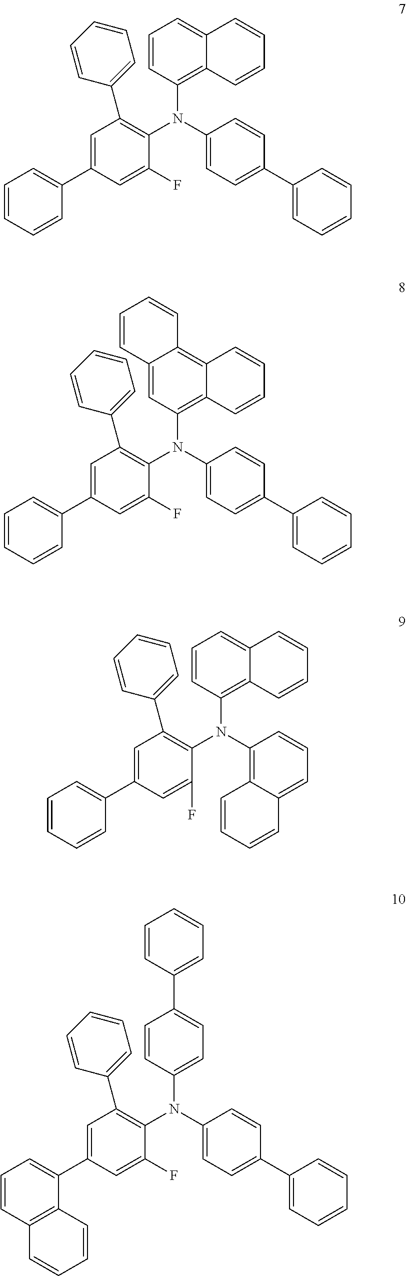 [SMILES: Fc1cc(-c2ccccc2)cc(-c2ccccc2)c1N(c1ccc(-c2ccccc2)cc1)c1cccc2ccccc12 ; Fc1cc(-c2ccccc2)cc(-c2ccccc2)c1N(c1ccc(-c2ccccc2)cc1)c1cc2ccccc2c2ccccc12 ; Fc1cc(-c2ccccc2)cc(-c2ccccc2)c1N(c1cccc2ccccc12)c1cccc2ccccc12 ; Fc1cc(-c2cccc3ccccc23)cc(-c2ccccc2)c1N(c1ccc(-c2ccccc2)cc1)c1ccc(-c2ccccc2)cc1]

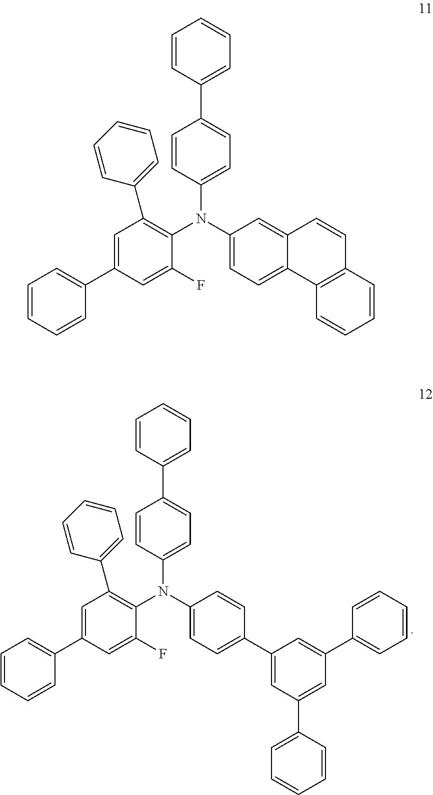 [SMILES: Fc1cc(-c2ccccc2)cc(-c2ccccc2)c1N(c1ccc(-c2ccccc2)cc1)c1ccc2c(ccc3ccccc32)c1 ; Fc1cc(-c2ccccc2)cc(-c2ccccc2)c1N(c1ccc(-c2ccccc2)cc1)c1ccc(-c2cc(-c3ccccc3)cc(-c3ccccc3)c2)cc1]

8. The amine derivative of claim 1, wherein the amine derivative represented by Formula 1 is selected from Compounds 13 to 36:

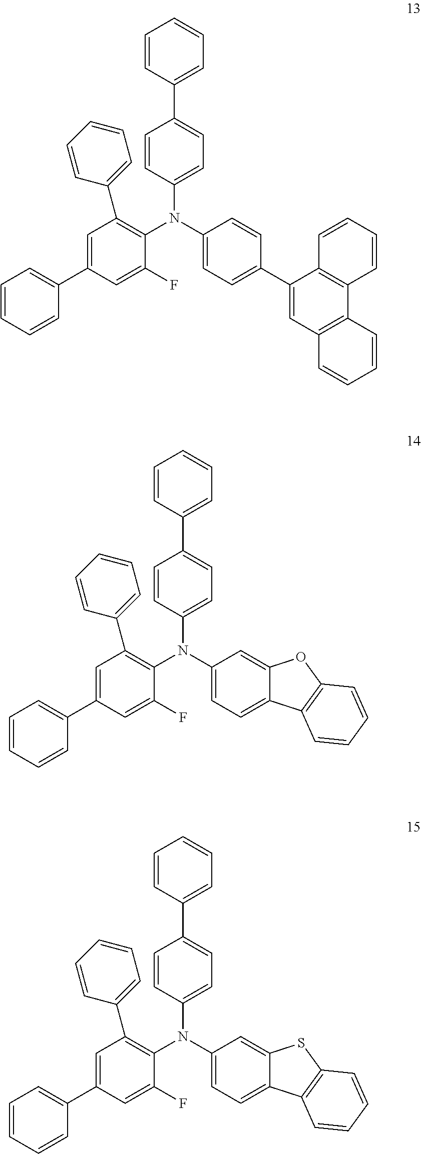 [SMILES: Fc1cc(-c2ccccc2)cc(-c2ccccc2)c1N(c1ccc(-c2ccccc2)cc1)c1ccc(-c2cc3ccccc3c3ccccc23)cc1 ; Fc1cc(-c2ccccc2)cc(-c2ccccc2)c1N(c1ccc(-c2ccccc2)cc1)c1ccc2c(c1)oc1ccccc12 ; Fc1cc(-c2ccccc2)cc(-c2ccccc2)c1N(c1ccc(-c2ccccc2)cc1)c1ccc2c(c1)sc1ccccc12]

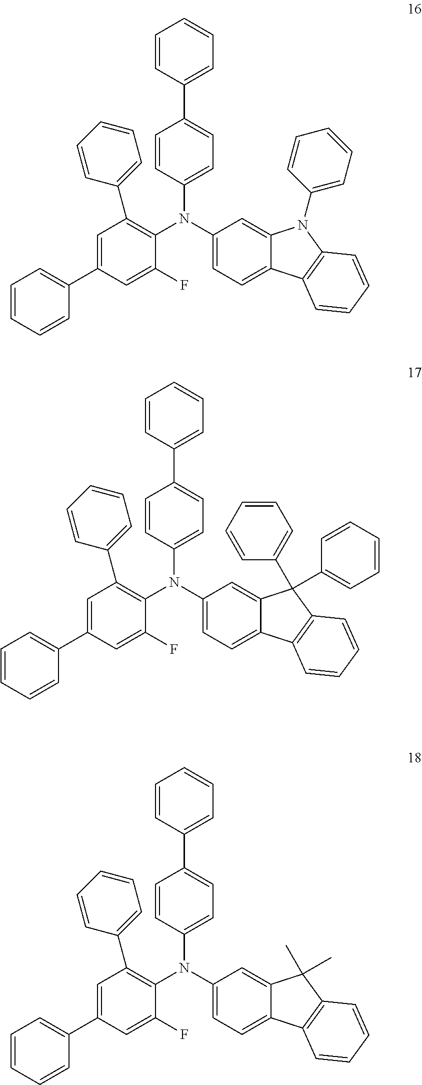 [SMILES: Fc1cc(-c2ccccc2)cc(-c2ccccc2)c1N(c1ccc(-c2ccccc2)cc1)c1ccc2c3ccccc3n(-c3ccccc3)c2c1 ; Fc1cc(-c2ccccc2)cc(-c2ccccc2)c1N(c1ccc(-c2ccccc2)cc1)c1ccc2c(c1)C(c1ccccc1)(c1ccccc1)c1ccccc1-2 ; CC1(C)c2ccccc2-c2ccc(N(c3ccc(-c4ccccc4)cc3)c3c(F)cc(-c4ccccc4)cc3-c3ccccc3)cc21]

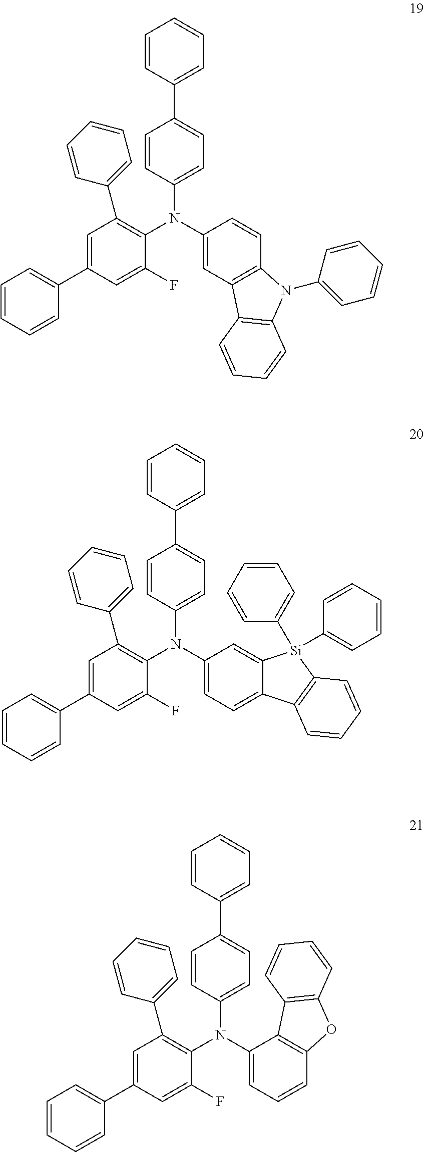 [SMILES: Fc1cc(-c2ccccc2)cc(-c2ccccc2)c1N(c1ccc(-c2ccccc2)cc1)c1ccc2c(c1)c1ccccc1n2-c1ccccc1 ; Fc1cc(-c2ccccc2)cc(-c2ccccc2)c1N(c1ccc(-c2ccccc2)cc1)c1ccc2c(c1)[Si](c1ccccc1)(c1ccccc1)c1ccccc1-2 ; Fc1cc(-c2ccccc2)cc(-c2ccccc2)c1N(c1ccc(-c2ccccc2)cc1)c1cccc2oc3ccccc3c12]

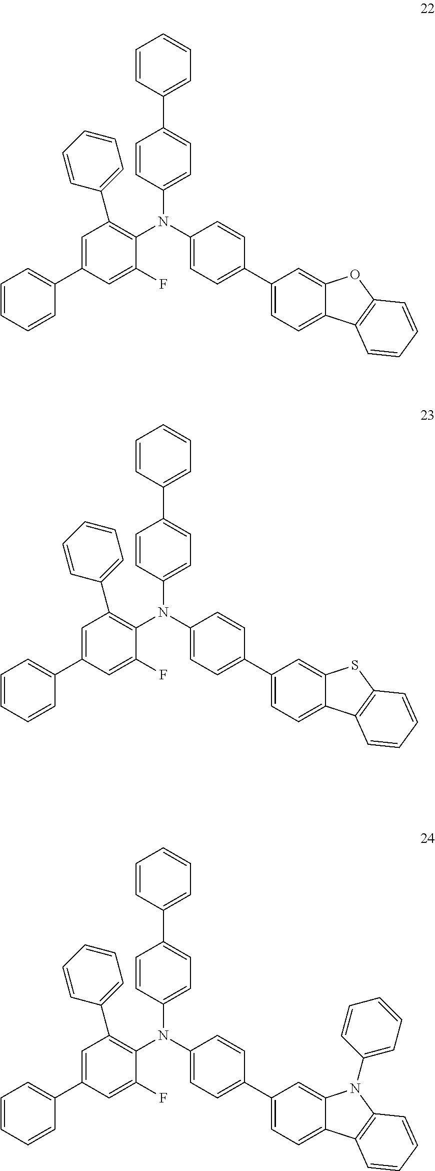 [SMILES: Fc1cc(-c2ccccc2)cc(-c2ccccc2)c1N(c1ccc(-c2ccccc2)cc1)c1ccc(-c2ccc3c(c2)oc2ccccc23)cc1 ; Fc1cc(-c2ccccc2)cc(-c2ccccc2)c1N(c1ccc(-c2ccccc2)cc1)c1ccc(-c2ccc3c(c2)sc2ccccc23)cc1 ; Fc1cc(-c2ccccc2)cc(-c2ccccc2)c1N(c1ccc(-c2ccccc2)cc1)c1ccc(-c2ccc3c4ccccc4n(-c4ccccc4)c3c2)cc1]

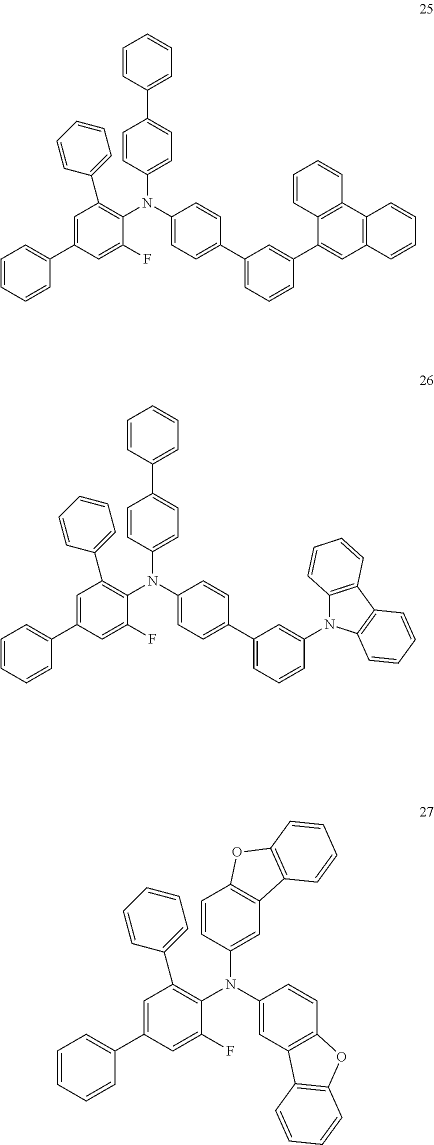 [SMILES: Fc1cc(-c2ccccc2)cc(-c2ccccc2)c1N(c1ccc(-c2ccccc2)cc1)c1ccc(-c2cccc(-c3cc4ccccc4c4ccccc34)c2)cc1 ; Fc1cc(-c2ccccc2)cc(-c2ccccc2)c1N(c1ccc(-c2ccccc2)cc1)c1ccc(-c2cccc(-n3c4ccccc4c4ccccc43)c2)cc1 ; Fc1cc(-c2ccccc2)cc(-c2ccccc2)c1N(c1ccc2oc3ccccc3c2c1)c1ccc2oc3ccccc3c2c1]

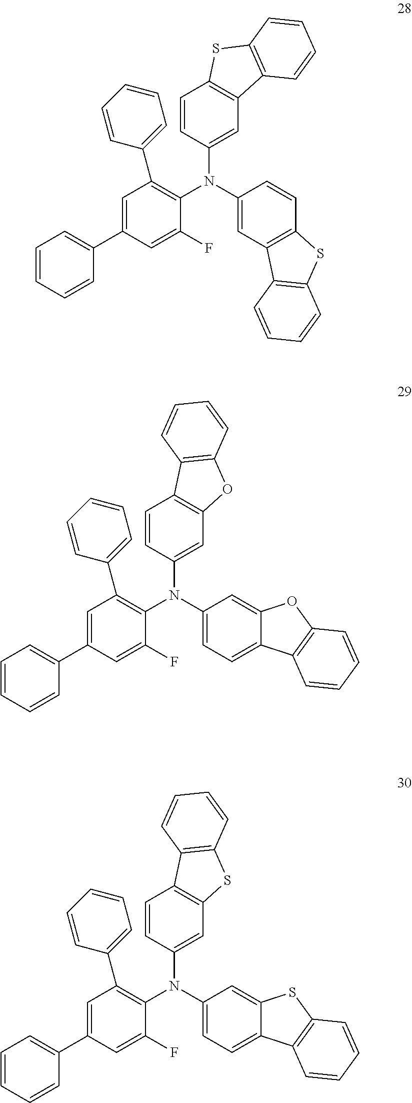 [SMILES: Fc1cc(-c2ccccc2)cc(-c2ccccc2)c1N(c1ccc2sc3ccccc3c2c1)c1ccc2sc3ccccc3c2c1 ; Fc1cc(-c2ccccc2)cc(-c2ccccc2)c1N(c1ccc2c(c1)oc1ccccc12)c1ccc2c(c1)oc1ccccc12 ; Fc1cc(-c2ccccc2)cc(-c2ccccc2)c1N(c1ccc2c(c1)sc1ccccc12)c1ccc2c(c1)sc1ccccc12]

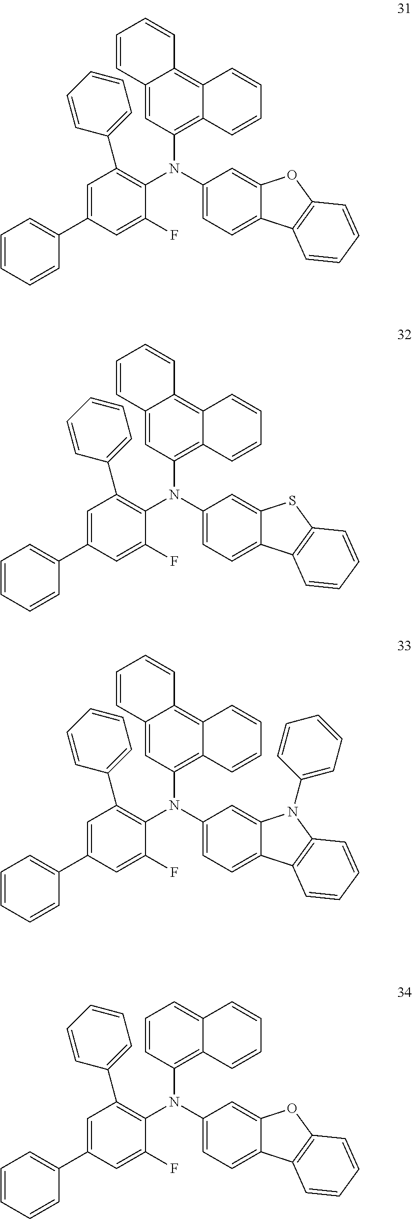 [SMILES: Fc1cc(-c2ccccc2)cc(-c2ccccc2)c1N(c1ccc2c(c1)oc1ccccc12)c1cc2ccccc2c2ccccc12 ; Fc1cc(-c2ccccc2)cc(-c2ccccc2)c1N(c1ccc2c(c1)sc1ccccc12)c1cc2ccccc2c2ccccc12 ; Fc1cc(-c2ccccc2)cc(-c2ccccc2)c1N(c1ccc2c3ccccc3n(-c3ccccc3)c2c1)c1cc2ccccc2c2ccccc12 ; Fc1cc(-c2ccccc2)cc(-c2ccccc2)c1N(c1ccc2c(c1)oc1ccccc12)c1cccc2ccccc12]

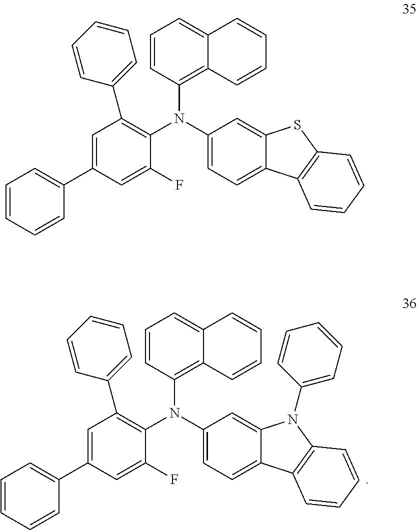 [SMILES: Fc1cc(-c2ccccc2)cc(-c2ccccc2)c1N(c1ccc2c(c1)sc1ccccc12)c1cccc2ccccc12 ; Fc1cc(-c2ccccc2)cc(-c2ccccc2)c1N(c1ccc2c3ccccc3n(-c3ccccc3)c2c1)c1cccc2ccccc12]

9. The amine derivative of claim 1, wherein the amine derivative represented by Formula 1 is selected from Compounds 37 to 52:

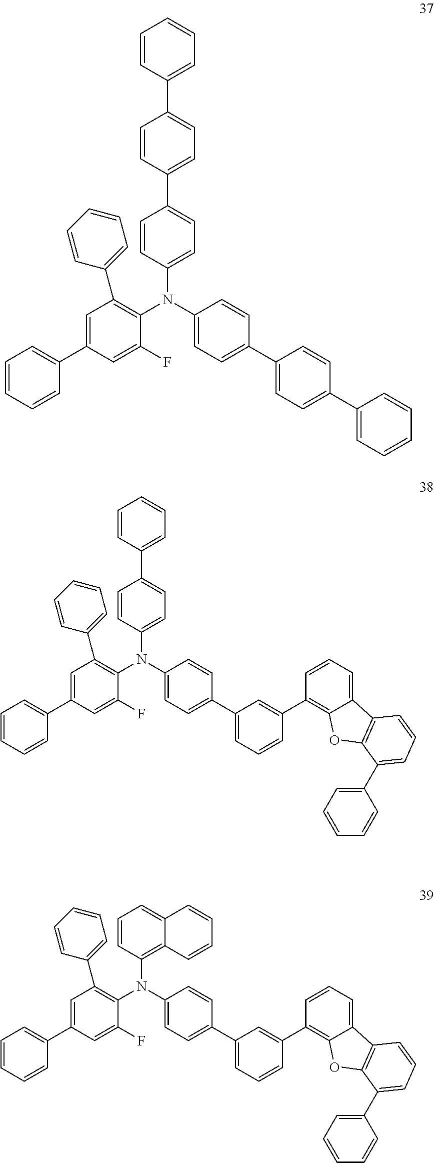 [SMILES: Fc1cc(-c2ccccc2)cc(-c2ccccc2)c1N(c1ccc(-c2ccc(-c3ccccc3)cc2)cc1)c1ccc(-c2ccc(-c3ccccc3)cc2)cc1 ; Fc1cc(-c2ccccc2)cc(-c2ccccc2)c1N(c1ccc(-c2ccccc2)cc1)c1ccc(-c2cccc(-c3cccc4c3oc3c(-c5ccccc5)cccc34)c2)cc1 ; Fc1cc(-c2ccccc2)cc(-c2ccccc2)c1N(c1ccc(-c2cccc(-c3cccc4c3oc3c(-c5ccccc5)cccc34)c2)cc1)c1cccc2ccccc12]

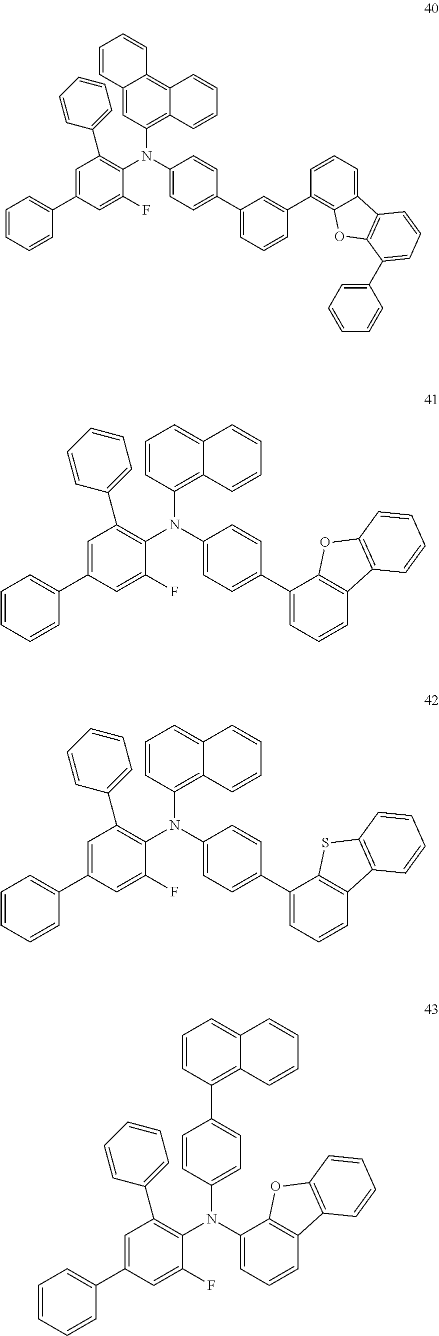 [SMILES: Fc1cc(-c2ccccc2)cc(-c2ccccc2)c1N(c1ccc(-c2cccc(-c3cccc4c3oc3c(-c5ccccc5)cccc34)c2)cc1)c1cc2ccccc2c2ccccc12 ; Fc1cc(-c2ccccc2)cc(-c2ccccc2)c1N(c1ccc(-c2cccc3c2oc2ccccc23)cc1)c1cccc2ccccc12 ; Fc1cc(-c2ccccc2)cc(-c2ccccc2)c1N(c1ccc(-c2cccc3c2sc2ccccc23)cc1)c1cccc2ccccc12 ; Fc1cc(-c2ccccc2)cc(-c2ccccc2)c1N(c1ccc(-c2cccc3ccccc23)cc1)c1cccc2c1oc1ccccc12]

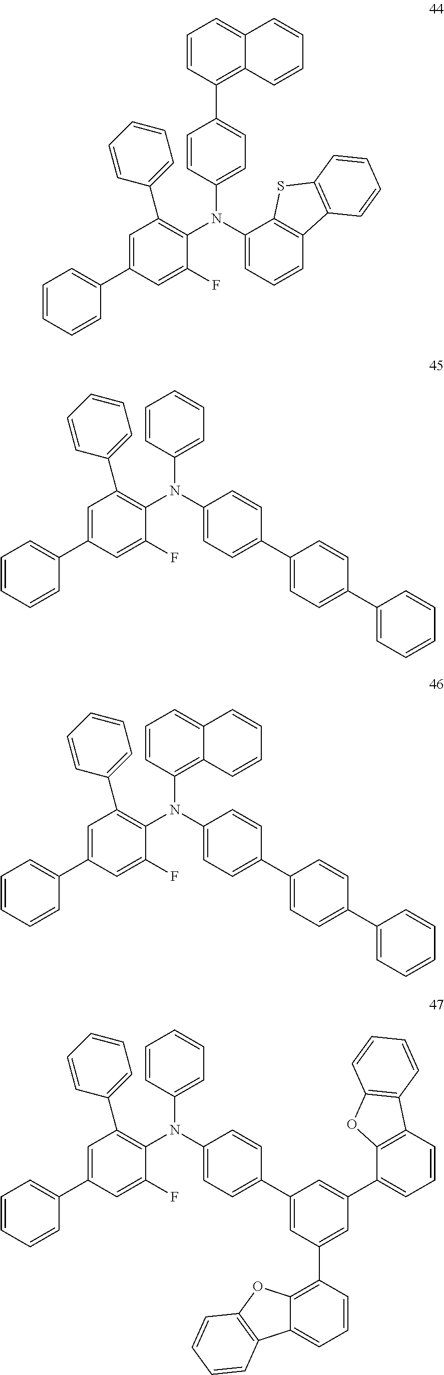 [SMILES: Fc1cc(-c2ccccc2)cc(-c2ccccc2)c1N(c1ccc(-c2cccc3ccccc23)cc1)c1cccc2c1sc1ccccc12 ; Fc1cc(-c2ccccc2)cc(-c2ccccc2)c1N(c1ccccc1)c1ccc(-c2ccc(-c3ccccc3)cc2)cc1 ; Fc1cc(-c2ccccc2)cc(-c2ccccc2)c1N(c1ccc(-c2ccc(-c3ccccc3)cc2)cc1)c1cccc2ccccc12 ; Fc1cc(-c2ccccc2)cc(-c2ccccc2)c1N(c1ccccc1)c1ccc(-c2cc(-c3cccc4c3oc3ccccc34)cc(-c3cccc4c3oc3ccccc34)c2)cc1]

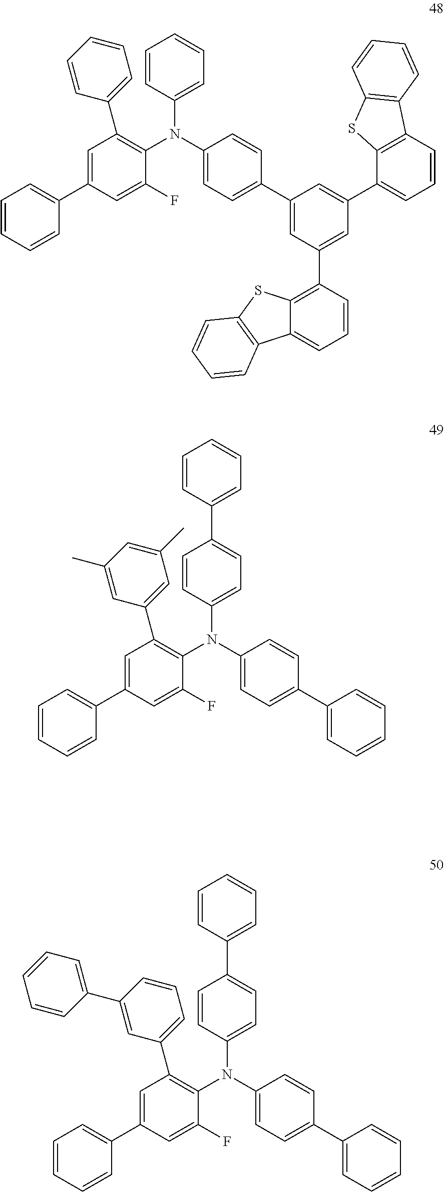 [SMILES: Fc1cc(-c2ccccc2)cc(-c2ccccc2)c1N(c1ccccc1)c1ccc(-c2cc(-c3cccc4c3sc3ccccc34)cc(-c3cccc4c3sc3ccccc34)c2)cc1 ; Cc1cc(C)cc(-c2cc(-c3ccccc3)cc(F)c2N(c2ccc(-c3ccccc3)cc2)c2ccc(-c3ccccc3)cc2)c1 ; Fc1cc(-c2ccccc2)cc(-c2cccc(-c3ccccc3)c2)c1N(c1ccc(-c2ccccc2)cc1)c1ccc(-c2ccccc2)cc1]

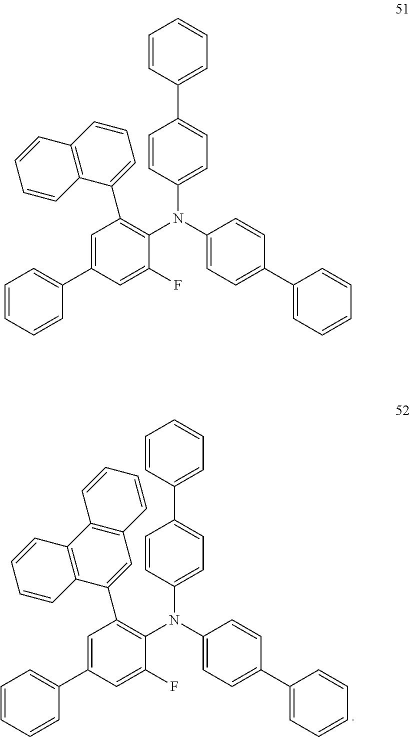 [SMILES: Fc1cc(-c2ccccc2)cc(-c2cccc3ccccc23)c1N(c1ccc(-c2ccccc2)cc1)c1ccc(-c2ccccc2)cc1 ; Fc1cc(-c2ccccc2)cc(-c2cc3ccccc3c3ccccc23)c1N(c1ccc(-c2ccccc2)cc1)c1ccc(-c2ccccc2)cc1]

10. An organic electroluminescent device, comprising:
an anode;
an emission layer on the anode; and
at least one layer between the anode and the emission layer,
wherein the emission layer or the at least one layer comprises an amine derivative represented by Formula 1: 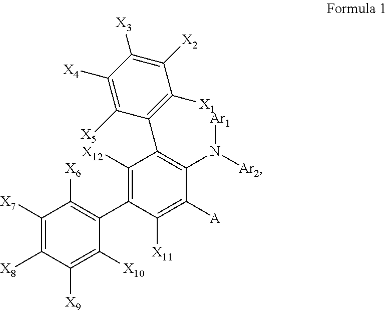
wherein:
X₁ to X₁₂ are each independently selected from hydrogen, deuterium, a halogen atom, a substituted or unsubstituted alkyl group having 1 to 15 carbon atoms, a substituted or unsubstituted aryl group having 6 to 36 carbon atoms for forming a ring, and a substituted or unsubstituted heteroaryl group having 1 to 36 carbon atoms for forming a ring, wherein a plurality of adjacent groups selected from X₁ to X₁₂ are optionally coupled to each other to form a condensed ring group;
Ar₁ and Ar₂ are each independently selected from a substituted or unsubstituted aryl group having 6 to 36 carbon atoms for forming a ring, and a substituted or unsubstituted heteroaryl group having 1 to 36 carbon atoms for forming a ring, the aryl group excluding a pyrenyl group; and
A is a halogen atom.

11. The organic electroluminescent device of claim 10, wherein in the amine derivative represented by Formula 1, the Ar₁ and Ar₂ each independently comprise the aryl group having 6 to 36 carbon atoms for forming a ring,
wherein the aryl group is selected from the group consisting of a phenyl group, a naphthyl group, an anthracenyl group, a phenanthryl group, a biphenyl group, a terphenyl group, a quaterphenyl group, a quinquephenyl group, a sexiphenyl group, a fluorenyl group, a triphenylene group, a biphenylene group, a benzofluoranthenyl group, a chrysenyl group, a phenylnaphthyl group, a naphthylphenyl group, a phenylanthracenyl group, and combinations thereof.

12. The organic electroluminescent device of claim 10, wherein in the amine derivative represented by Formula 1, the Ar₁ and Ar₂ each independently comprise the heteroaryl group having 1 to 36 carbon atoms for forming a ring,
wherein the heteroaryl group is selected from the group consisting of a dibenzofuranyl group, a dibenzothienyl group, a carbazolyl group, a dibenzosilolyl group, a phenyldibenzofuranyl group, a phenyldibenzothienyl group, a phenylcarbazolyl group, and combinations thereof.

13. The organic electroluminescent device of claim 10, wherein in the amine derivative represented by Formula 1, at least one selected from the Ar₁ and Ar₂ is selected from a substituted or unsubstituted dibenzofuranyl group, a substituted or unsubstituted dibenzothienyl group, a substituted or unsubstituted carbazolyl group, a substituted or unsubstituted dibenzosilolyl group, and an aryl group substituted with at least one selected from a dibenzofuranyl group, a dibenzothienyl group, a carbazolyl group, and a dibenzosilolyl group.

14. The organic electroluminescent device of claim 10, wherein in the amine derivative represented by Formula 1, the A is a fluorine atom.

15. The organic electroluminescent device of claim 10, wherein in the amine derivative represented by Formula 1, the X₁ to X₁₂ are each independently selected from hydrogen, deuterium, a methyl group, and a phenyl group, wherein a plurality of adjacent groups selected from X₁ to X₁₂ are optionally coupled to each other to form a condensed ring group.

16. The organic electroluminescent device of claim 10, wherein the amine derivative represented by Formula 1 comprises at least one selected from Compounds 1 to 12:

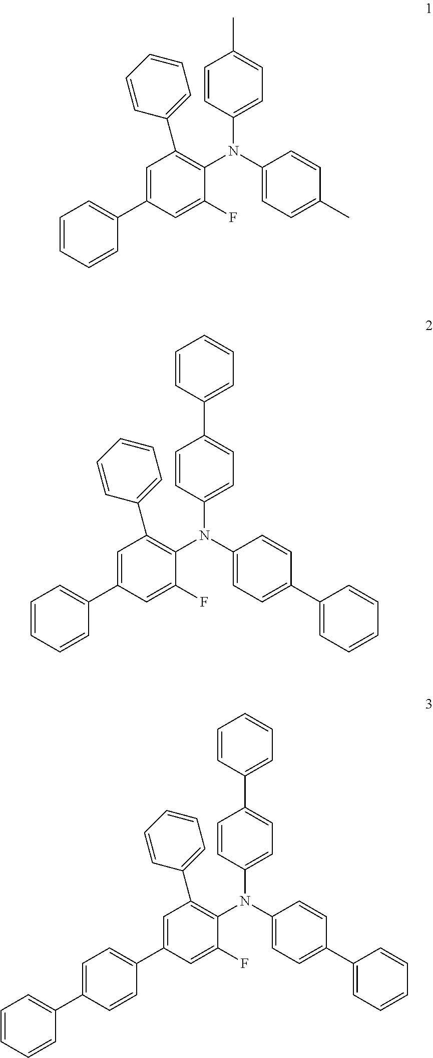 [SMILES: Cc1ccc(N(c2ccc(C)cc2)c2c(F)cc(-c3ccccc3)cc2-c2ccccc2)cc1 ; Fc1cc(-c2ccccc2)cc(-c2ccccc2)c1N(c1ccc(-c2ccccc2)cc1)c1ccc(-c2ccccc2)cc1 ; Fc1cc(-c2ccc(-c3ccccc3)cc2)cc(-c2ccccc2)c1N(c1ccc(-c2ccccc2)cc1)c1ccc(-c2ccccc2)cc1]

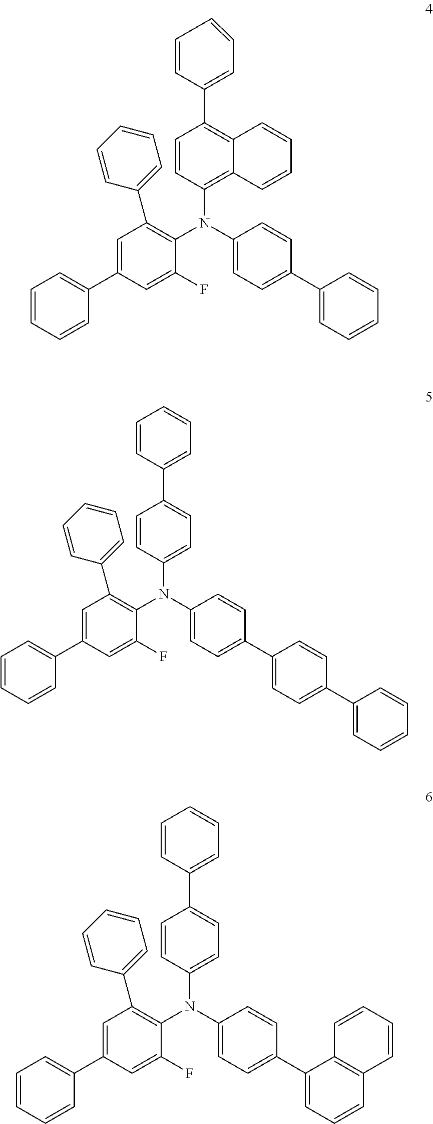 [SMILES: Fc1cc(-c2ccccc2)cc(-c2ccccc2)c1N(c1ccc(-c2ccccc2)cc1)c1ccc(-c2ccccc2)c2ccccc12 ; Fc1cc(-c2ccccc2)cc(-c2ccccc2)c1N(c1ccc(-c2ccccc2)cc1)c1ccc(-c2ccc(-c3ccccc3)cc2)cc1 ; Fc1cc(-c2ccccc2)cc(-c2ccccc2)c1N(c1ccc(-c2ccccc2)cc1)c1ccc(-c2cccc3ccccc23)cc1]

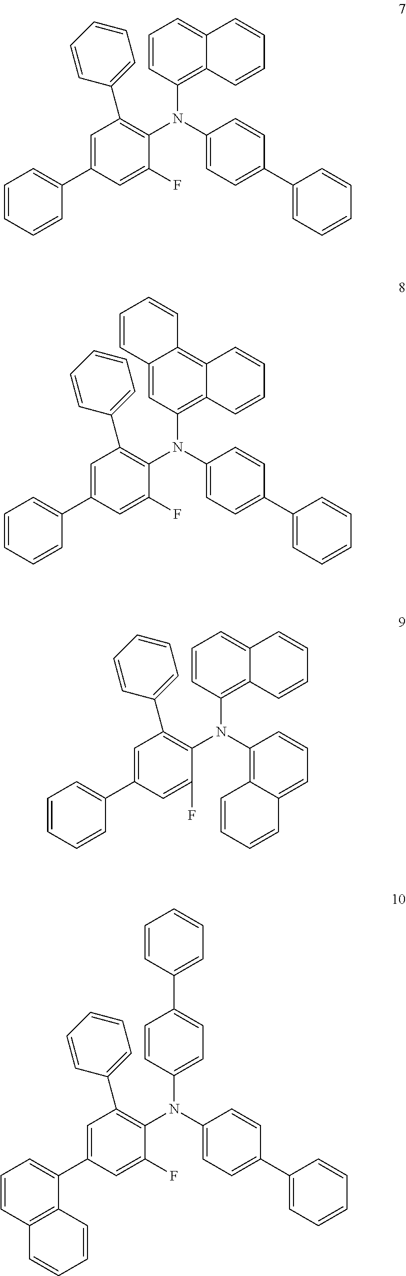 [SMILES: Fc1cc(-c2ccccc2)cc(-c2ccccc2)c1N(c1ccc(-c2ccccc2)cc1)c1cccc2ccccc12 ; Fc1cc(-c2ccccc2)cc(-c2ccccc2)c1N(c1ccc(-c2ccccc2)cc1)c1cc2ccccc2c2ccccc12 ; Fc1cc(-c2ccccc2)cc(-c2ccccc2)c1N(c1cccc2ccccc12)c1cccc2ccccc12 ; Fc1cc(-c2cccc3ccccc23)cc(-c2ccccc2)c1N(c1ccc(-c2ccccc2)cc1)c1ccc(-c2ccccc2)cc1]

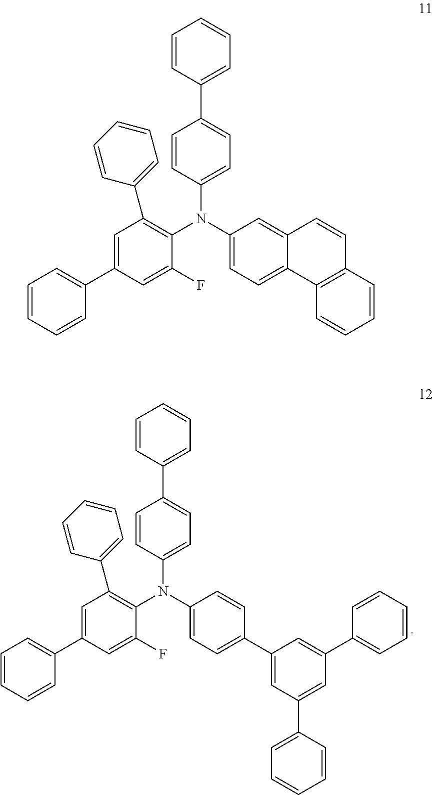 [SMILES: Fc1cc(-c2ccccc2)cc(-c2ccccc2)c1N(c1ccc(-c2ccccc2)cc1)c1ccc2c(ccc3ccccc32)c1 ; Fc1cc(-c2ccccc2)cc(-c2ccccc2)c1N(c1ccc(-c2ccccc2)cc1)c1ccc(-c2cc(-c3ccccc3)cc(-c3ccccc3)c2)cc1]

17. The organic electroluminescent device of claim 10, wherein the amine derivative represented by Formula 1 comprises at least one selected from Compounds 13 to 36:

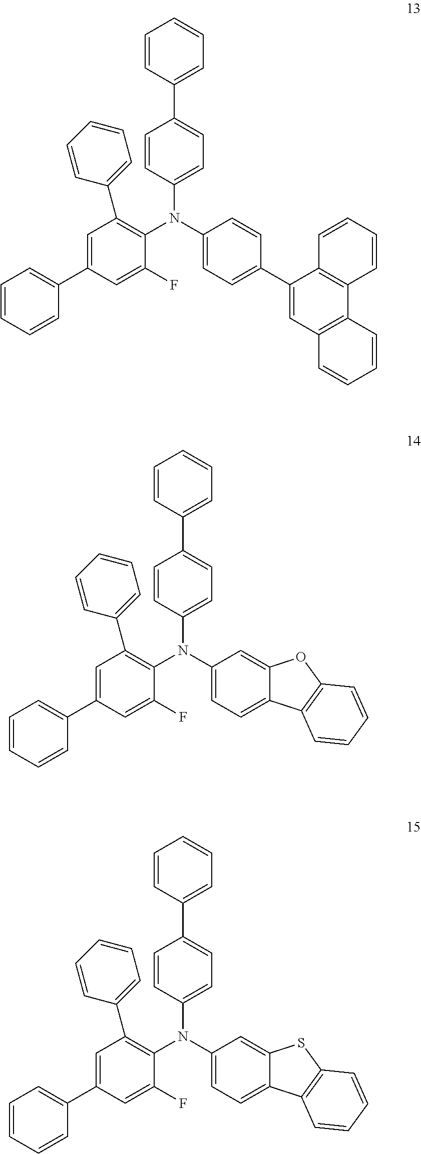 [SMILES: Fc1cc(-c2ccccc2)cc(-c2ccccc2)c1N(c1ccc(-c2ccccc2)cc1)c1ccc(-c2cc3ccccc3c3ccccc23)cc1 ; Fc1cc(-c2ccccc2)cc(-c2ccccc2)c1N(c1ccc(-c2ccccc2)cc1)c1ccc2c(c1)oc1ccccc12 ; Fc1cc(-c2ccccc2)cc(-c2ccccc2)c1N(c1ccc(-c2ccccc2)cc1)c1ccc2c(c1)sc1ccccc12]

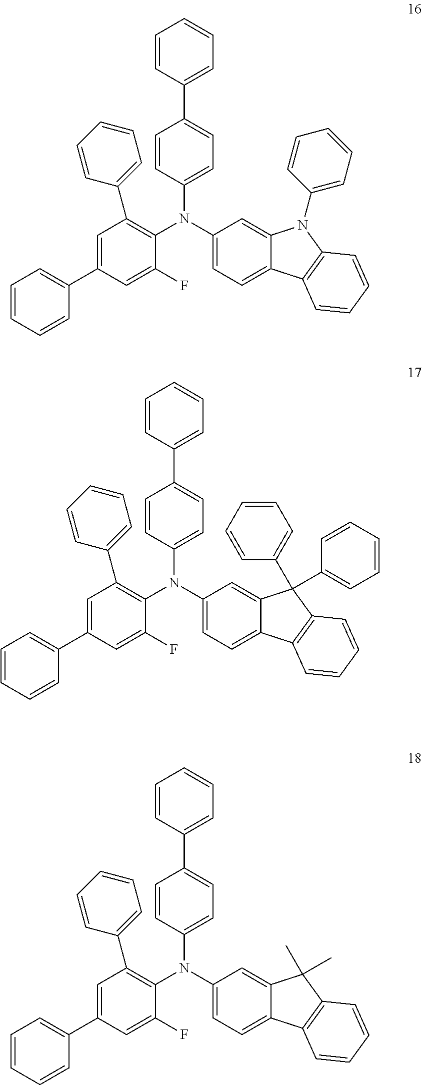 [SMILES: Fc1cc(-c2ccccc2)cc(-c2ccccc2)c1N(c1ccc(-c2ccccc2)cc1)c1ccc2c3ccccc3n(-c3ccccc3)c2c1 ; Fc1cc(-c2ccccc2)cc(-c2ccccc2)c1N(c1ccc(-c2ccccc2)cc1)c1ccc2c(c1)C(c1ccccc1)(c1ccccc1)c1ccccc1-2 ; CC1(C)c2ccccc2-c2ccc(N(c3ccc(-c4ccccc4)cc3)c3c(F)cc(-c4ccccc4)cc3-c3ccccc3)cc21]

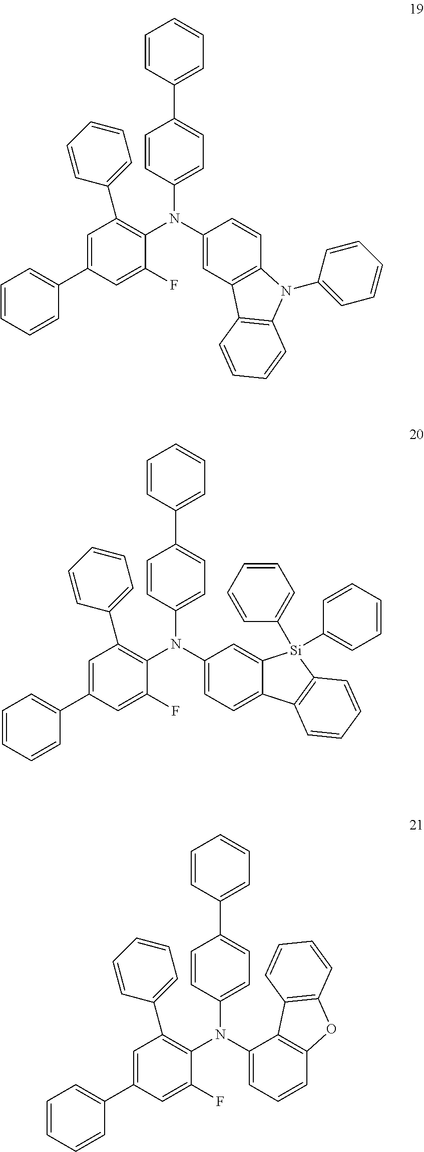 [SMILES: Fc1cc(-c2ccccc2)cc(-c2ccccc2)c1N(c1ccc(-c2ccccc2)cc1)c1ccc2c(c1)c1ccccc1n2-c1ccccc1 ; Fc1cc(-c2ccccc2)cc(-c2ccccc2)c1N(c1ccc(-c2ccccc2)cc1)c1ccc2c(c1)[Si](c1ccccc1)(c1ccccc1)c1ccccc1-2 ; Fc1cc(-c2ccccc2)cc(-c2ccccc2)c1N(c1ccc(-c2ccccc2)cc1)c1cccc2oc3ccccc3c12]

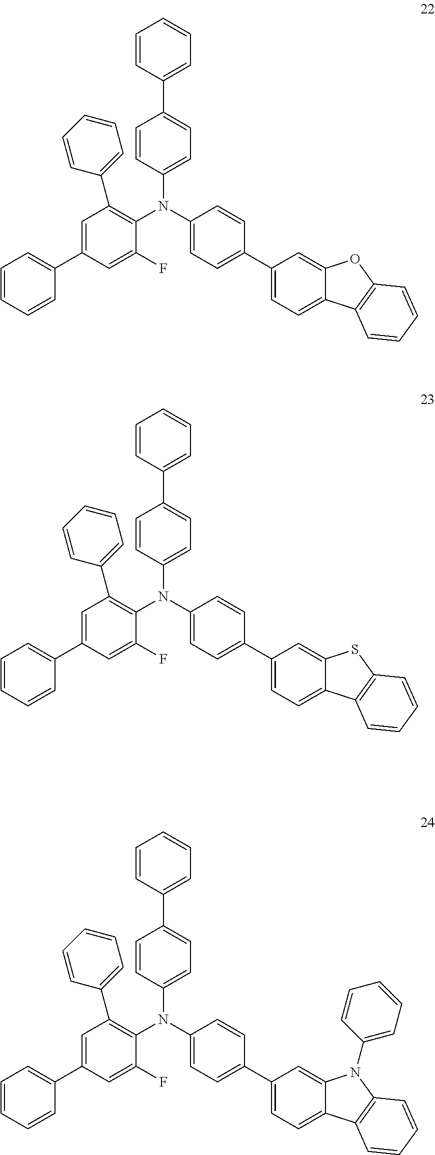 [SMILES: Fc1cc(-c2ccccc2)cc(-c2ccccc2)c1N(c1ccc(-c2ccccc2)cc1)c1ccc(-c2ccc3c(c2)oc2ccccc23)cc1 ; Fc1cc(-c2ccccc2)cc(-c2ccccc2)c1N(c1ccc(-c2ccccc2)cc1)c1ccc(-c2ccc3c(c2)sc2ccccc23)cc1 ; Fc1cc(-c2ccccc2)cc(-c2ccccc2)c1N(c1ccc(-c2ccccc2)cc1)c1ccc(-c2ccc3c4ccccc4n(-c4ccccc4)c3c2)cc1]

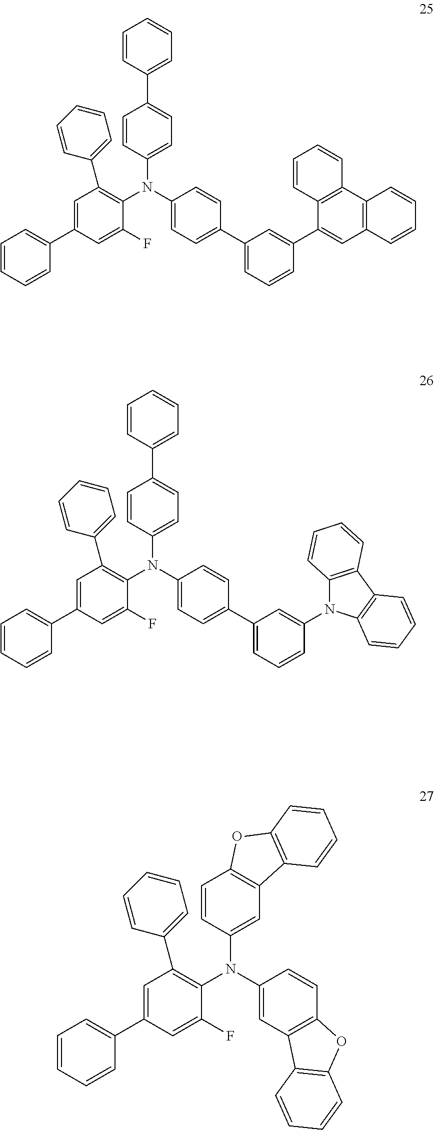 [SMILES: Fc1cc(-c2ccccc2)cc(-c2ccccc2)c1N(c1ccc(-c2ccccc2)cc1)c1ccc(-c2cccc(-c3cc4ccccc4c4ccccc34)c2)cc1 ; Fc1cc(-c2ccccc2)cc(-c2ccccc2)c1N(c1ccc(-c2ccccc2)cc1)c1ccc(-c2cccc(-n3c4ccccc4c4ccccc43)c2)cc1 ; Fc1cc(-c2ccccc2)cc(-c2ccccc2)c1N(c1ccc2oc3ccccc3c2c1)c1ccc2oc3ccccc3c2c1]

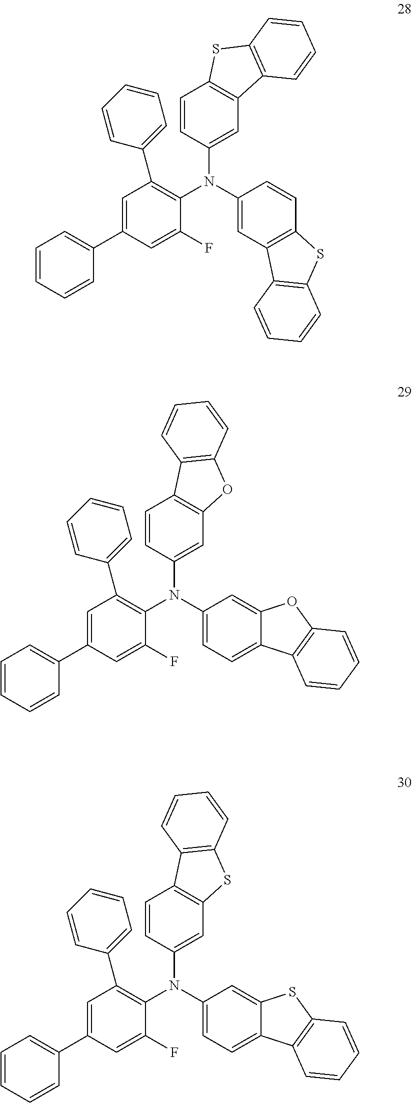 [SMILES: Fc1cc(-c2ccccc2)cc(-c2ccccc2)c1N(c1ccc2sc3ccccc3c2c1)c1ccc2sc3ccccc3c2c1 ; Fc1cc(-c2ccccc2)cc(-c2ccccc2)c1N(c1ccc2c(c1)oc1ccccc12)c1ccc2c(c1)oc1ccccc12 ; Fc1cc(-c2ccccc2)cc(-c2ccccc2)c1N(c1ccc2c(c1)sc1ccccc12)c1ccc2c(c1)sc1ccccc12]

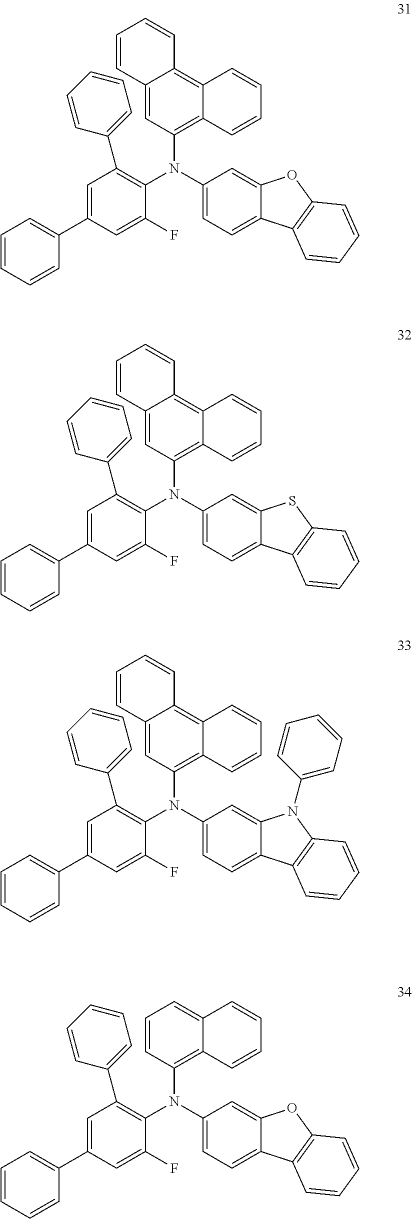 [SMILES: Fc1cc(-c2ccccc2)cc(-c2ccccc2)c1N(c1ccc2c(c1)oc1ccccc12)c1cc2ccccc2c2ccccc12 ; Fc1cc(-c2ccccc2)cc(-c2ccccc2)c1N(c1ccc2c(c1)sc1ccccc12)c1cc2ccccc2c2ccccc12 ; Fc1cc(-c2ccccc2)cc(-c2ccccc2)c1N(c1ccc2c3ccccc3n(-c3ccccc3)c2c1)c1cc2ccccc2c2ccccc12 ; Fc1cc(-c2ccccc2)cc(-c2ccccc2)c1N(c1ccc2c(c1)oc1ccccc12)c1cccc2ccccc12]

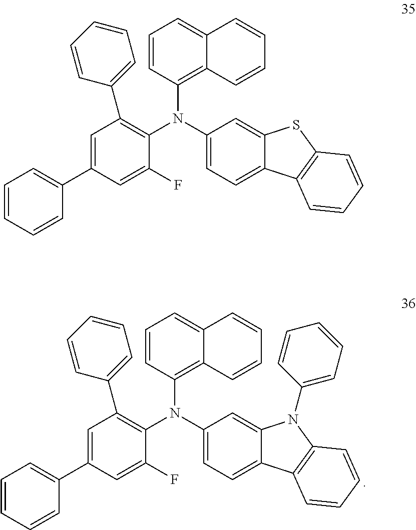 [SMILES: Fc1cc(-c2ccccc2)cc(-c2ccccc2)c1N(c1ccc2c(c1)sc1ccccc12)c1cccc2ccccc12 ; Fc1cc(-c2ccccc2)cc(-c2ccccc2)c1N(c1ccc2c3ccccc3n(-c3ccccc3)c2c1)c1cccc2ccccc12]

18. The organic electroluminescent device of claim 10, wherein the amine derivative represented by Formula 1 comprises at least one selected from Compounds 37 to 52:

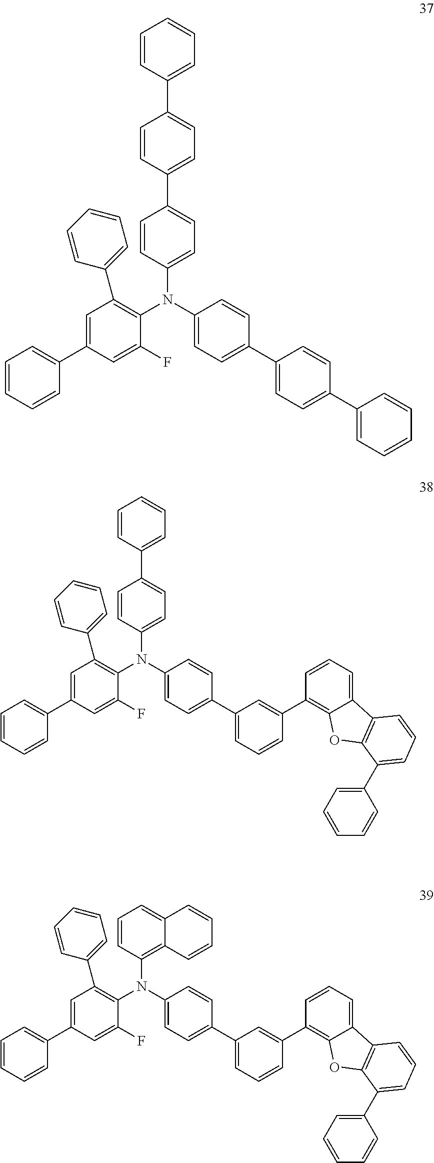 [SMILES: Fc1cc(-c2ccccc2)cc(-c2ccccc2)c1N(c1ccc(-c2ccc(-c3ccccc3)cc2)cc1)c1ccc(-c2ccc(-c3ccccc3)cc2)cc1 ; Fc1cc(-c2ccccc2)cc(-c2ccccc2)c1N(c1ccc(-c2ccccc2)cc1)c1ccc(-c2cccc(-c3cccc4c3oc3c(-c5ccccc5)cccc34)c2)cc1 ; Fc1cc(-c2ccccc2)cc(-c2ccccc2)c1N(c1ccc(-c2cccc(-c3cccc4c3oc3c(-c5ccccc5)cccc34)c2)cc1)c1cccc2ccccc12]

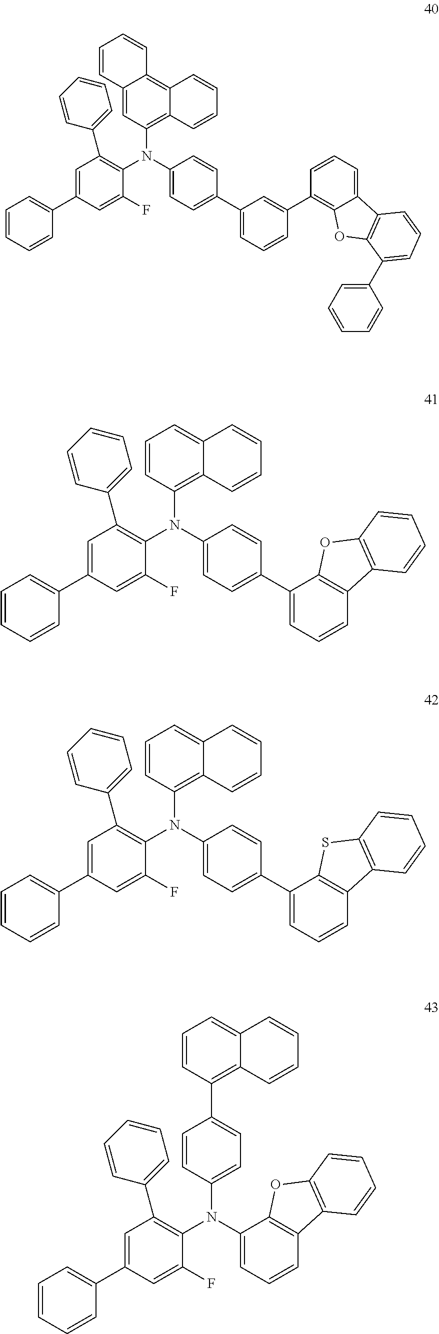 [SMILES: Fc1cc(-c2ccccc2)cc(-c2ccccc2)c1N(c1ccc(-c2cccc(-c3cccc4c3oc3c(-c5ccccc5)cccc34)c2)cc1)c1cc2ccccc2c2ccccc12 ; Fc1cc(-c2ccccc2)cc(-c2ccccc2)c1N(c1ccc(-c2cccc3c2oc2ccccc23)cc1)c1cccc2ccccc12 ; Fc1cc(-c2ccccc2)cc(-c2ccccc2)c1N(c1ccc(-c2cccc3c2sc2ccccc23)cc1)c1cccc2ccccc12 ; Fc1cc(-c2ccccc2)cc(-c2ccccc2)c1N(c1ccc(-c2cccc3ccccc23)cc1)c1cccc2c1oc1ccccc12]

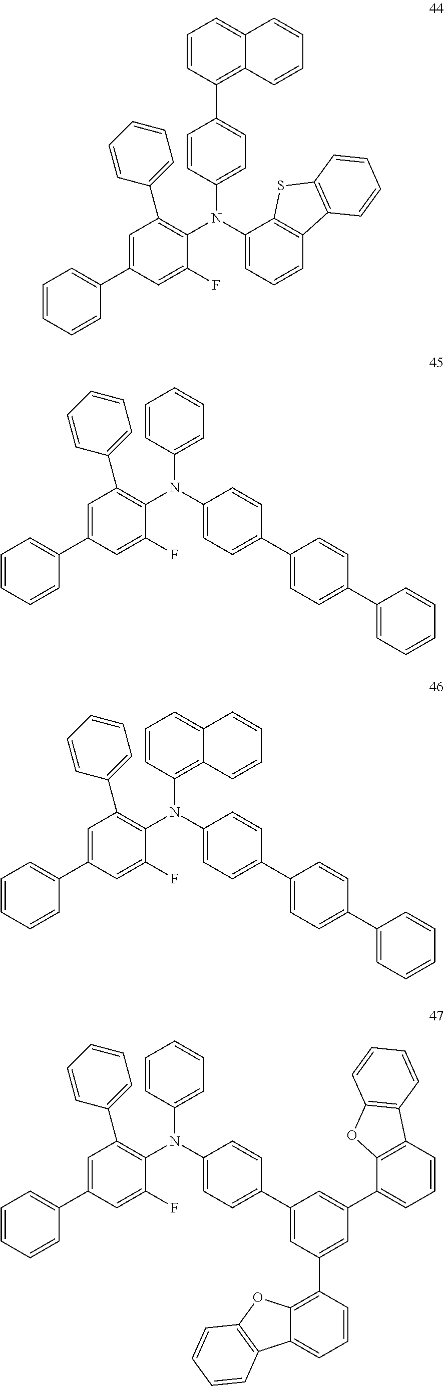 [SMILES: Fc1cc(-c2ccccc2)cc(-c2ccccc2)c1N(c1ccc(-c2cccc3ccccc23)cc1)c1cccc2c1sc1ccccc12 ; Fc1cc(-c2ccccc2)cc(-c2ccccc2)c1N(c1ccccc1)c1ccc(-c2ccc(-c3ccccc3)cc2)cc1 ; Fc1cc(-c2ccccc2)cc(-c2ccccc2)c1N(c1ccc(-c2ccc(-c3ccccc3)cc2)cc1)c1cccc2ccccc12 ; Fc1cc(-c2ccccc2)cc(-c2ccccc2)c1N(c1ccccc1)c1ccc(-c2cc(-c3cccc4c3oc3ccccc34)cc(-c3cccc4c3oc3ccccc34)c2)cc1]

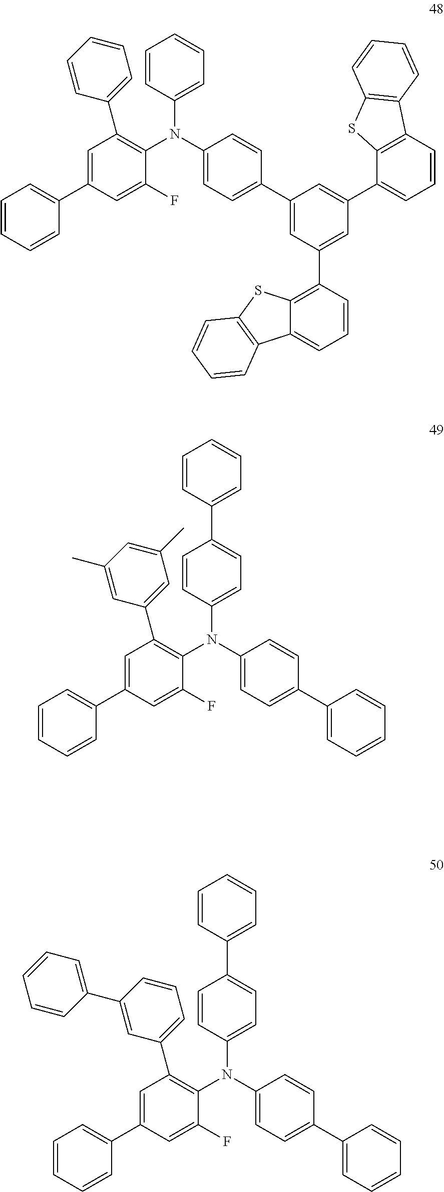 [SMILES: Fc1cc(-c2ccccc2)cc(-c2ccccc2)c1N(c1ccccc1)c1ccc(-c2cc(-c3cccc4c3sc3ccccc34)cc(-c3cccc4c3sc3ccccc34)c2)cc1 ; Cc1cc(C)cc(-c2cc(-c3ccccc3)cc(F)c2N(c2ccc(-c3ccccc3)cc2)c2ccc(-c3ccccc3)cc2)c1 ; Fc1cc(-c2ccccc2)cc(-c2cccc(-c3ccccc3)c2)c1N(c1ccc(-c2ccccc2)cc1)c1ccc(-c2ccccc2)cc1]

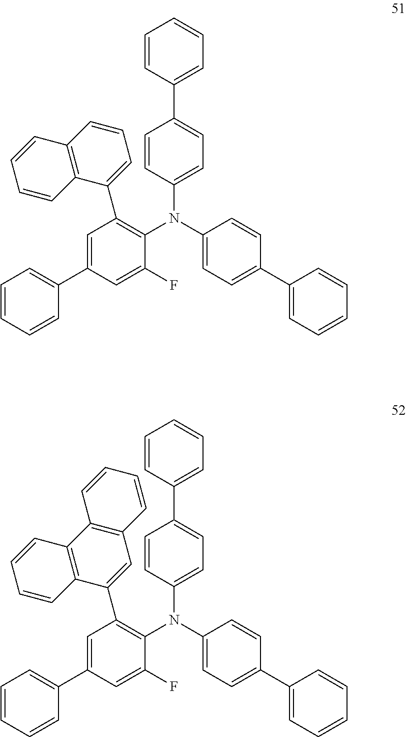 [SMILES: Fc1cc(-c2ccccc2)cc(-c2cccc3ccccc23)c1N(c1ccc(-c2ccccc2)cc1)c1ccc(-c2ccccc2)cc1 ; Fc1cc(-c2ccccc2)cc(-c2cc3ccccc3c3ccccc23)c1N(c1ccc(-c2ccccc2)cc1)c1ccc(-c2ccccc2)cc1]

19. The organic electroluminescent device of claim 10, wherein:
the at least one layer comprises a hole injection layer and a hole transport layer; and
at least one selected from the hole injection layer and the hole transport layer comprises the amine derivative represented by Formula 1.

20. The organic electroluminescent device of claim 19, wherein:
the hole injection layer is on the anode;
the hole transport layer is on the hole injection layer; and
the hole transport layer comprises the amine derivative represented by Formula 1.
